(19) United States
(12) Patent Application Publication
(10) Pub. No.: US 2026/0264055 A1
(43) Pub. Date: Sep. 10, 2026

(54) CATALYSTS FOR OXIDATIVE DEHYDROGENATION

(71) Applicant: NOVA CHEMICALS (INTERNATIONAL) S.A., Fribourg (CH)

(72) Inventor: Jared Taylor, Calgary (CA)

(21) Appl. No.: 19/164,034

(22) PCT Filed: Mar. 12, 2024
(86) PCT No.: PCT/IB2024/052375

§ 371 Date: Sep. 10, 2025

Related U.S. Application Data
(60) Provisional application No. 63/489,902, filed on Mar. 13, 2023.

(51) Int. Cl. B01J 23/31; B01J 35/61; B01J 35/63; B01J 37/00; C07C 5/48; C08F 10/02

(52) U.S. Cl. CPC B01J 23/31; B01J 35/613; B01J 35/633; B01J 37/0036; B01J 37/0045; C07C 5/48; C08F 10/02; B01J 2235/15; B01J 2235/30; C07C 2523/31

(57) ABSTRACT

A catalyst and methods for making the catalyst are provided. An exemplary catalyst includes the formula: MoaVbBicMdOx. In this formula, M is Ta or Nb, a is 1.0, b is 0.01 to 0.5, c is 0.005 to 0.2, d is 0.005 to 0.1, and x is the number of oxygen atoms necessary to render the catalyst electrically neutral. The values a, b, c, and d are determined based on the 5 amount of each starting material used to form the catalyst. The catalysts provided herein may be suitable as catalyst in oxidative dehydrogenation reactions, such as in the oxidative dehydrogenation of ethane.

### TECHNICAL FIELD

The present disclosure relates generally to catalysts and catalyst materials for oxidative dehydrogenation (ODH) of alkanes such as ethane. More specifically, the catalysts disclosed herein contain molybdenum (Mo); vanadium (V); bismuth (Bi); tantalum (Ta) or niobium (Nb), or both; and oxygen (O).

### BACKGROUND ART

Olefins like ethylene, propylene, and butylene, are basic building blocks for a variety of commercially valuable polymers. Since naturally occurring sources of olefins do not exist in commercial quantities, polymer producers rely on methods for converting the more abundant lower alkanes into olefins. The method of choice for today's commercial scale producers is steam cracking, a highly endothermic process where steam-diluted hydrocarbons are subjected very briefly to a temperature of at least 600° C. The fuel demand to produce the required temperature and the need for equipment that can withstand that temperature add significantly to the overall cost. In addition, the high temperature promotes the formation of coke, which accumulates within the system, resulting in the need for costly periodic reactor shutdowns for maintenance and coke removal.

Selective oxidation processes, such as oxidative dehydrogenation (ODH), are an alternative to steam cracking that are exothermic and produce little or no coke. In ODH, a lower alkane, such as ethane, is mixed with oxygen in the presence of a catalyst and optionally an inert diluent, such as carbon dioxide or nitrogen or steam, which may be performed at temperatures as low as 300° C., to produce the corresponding alkene. Various other oxidation products may be produced in this process, including carbon dioxide and acetic acid, among others. ODH suffers from lower conversion rates when compared to steam cracking, a fact that when combined with lower selectivity may have prevented ODH from achieving widespread commercial implementation. There is a need for a catalyst for an ODH of ethane process with high ethylene selectivity, activity, and longevity.

### SUMMARY OF INVENTION

Provided herein is a catalyst including the formula MoₐV_{b}Bi_{c}M_{d}Oₓ, wherein M is Ta or Nb, or a mixture thereof, a is 1.0; b is 0.01 to 0.5; c is 0.005 to 0.2; d is 0.005 to 0.1; and x is the number of oxygen atoms necessary to render the catalyst electrically neutral, wherein the values of a, b, c, and d are determined based on the amount of each starting material used to form the catalyst. In some embodiments, the values of a, b, c, and d are also determined by elemental analysis.

In some embodiments, b is 0.2 to 0.4; c is 0.01 to 0.07; and d is 0.01 to 0.07. In some embodiments, b is 0.30 to 0.35; c is 0.04 to 0.05; and d is 0.03 to 0.05. In some embodiments, b is 0.2 to 0.3; c is 0.05 to 0.07; and d is 0.02 to 0.04. In some embodiments, b is 0.3 to 0.4; c is 0.05 to 0.07; and d is 0.03 to 0.05.

In some embodiments, the catalyst has a formula selected from Mo₁V_{0.31}Bi_{0.04}M_{0.05}Oₓ, Mo₁V_{0.31}Bi_{0.05}M_{0.05}Oₓ, Mo₁V_{0.31}Bi_{0.06}M_{0.05}Oₓ, Mo₁V_{0.32}Bi_{0.05}M_{0.05}Oₓ, Mo₁V_{0.32}Bi_{0.04}M_{0.03}Oₓ, Mo₁V_{0.33}Bi_{0.05}M_{0.04}Oₓ, Mo₁V_{0.26}Bi_{0.06}M_{0.03}Oₓ, Mo₁V_{0.33}Bi_{0.06}M_{0.04}Oₓ, and Mo₁V_{0.26}Bi_{0.05}M_{0.05}Oₓ.

In some embodiments, the catalyst has a formula selected from Mo₁V_{0.32}Bi_{0.04}Ta_{0.03}Oₓ, Mo₁V_{0.33}Bi_{0.05}Ta_{0.04}Oₓ, Mo₁V_{0.26}Bi_{0.06}Ta_{0.03}Oₓ, Mo₁V_{0.31}Bi_{0.06}Nb_{0.01}Oₓ, Mo₁V_{0.33}Bi_{0.06}Ta_{0.04}Oₓ, and Mo₁V_{0.26}Bi_{0.05}Ta_{0.05}Oₓ.

In some embodiments, the catalyst has a formula selected from Mo₁V_{0.32} Bi_{0.04}Ta_{0.03}Oₓ, Mo₁V_{0.33}Bi_{0.05}Ta_{0.04}Oₓ, Mo₁V_{0.26}Bi_{0.05}Ta_{0.05}Oₓ, and Mo₁V_{0.26}Bi_{0.06}Ta_{0.03}Oₓ, wherein the formula is determined by energy dispersive X-ray spectroscopy (EDX).

In some embodiments, the catalyst as a pore volume from 0.02 cm³/g to 0.25 cm³/g, as determined by a nitrogen physisorption analysis with a Barrett-Joyner-Halenda (BJH) model.

In some embodiments, a pore volume from 0.1 cm³/g to 0.2 cm³/g, as determined by a nitrogen physisorption analysis with a Barrett-Joyner-Halenda (BJH) model.

In some embodiments, the catalyst has a Brunauer-Emmett-Teller (BET) surface area from 5 m²/g to 60 m²/g, as determined by a nitrogen physisorption analysis.

In some embodiments, the catalyst has a Brunauer-Emmett-Teller (BET) surface area from 10 m²/g to 25 m²/g, as determined by a nitrogen physisorption analysis.

The present disclosure also provides a catalyst material. In some embodiments, a catalyst material includes the catalyst described herein and a catalyst support or carrier. In some embodiments, the catalyst support or carrier is selected from the group consisting of precipitated synthetic silica, fumed synthetic silica, silica-alumina, α-alumina, γ-alumina, titania, WO₃—ZrO₂, silicon carbide, MgAl spinel, calcium aluminate, zirconia, and boron nitride. In some embodiments, the catalyst support or carrier is α-alumina.

In some embodiments, the catalyst has an ethylene selectivity of at least 90% at an ethane conversion of at least 45% in an oxidative dehydrogenation reaction of ethane.

In some embodiments, the catalyst has a 45% ethane conversion temperature from 300° C. to 420° C. in an oxidative dehydrogenation reaction of ethane. In some embodiments, the catalyst has a 45% ethane conversion temperature from 350° C. to 400° C. in an oxidative dehydrogenation reaction of ethane.

In some embodiments, the catalyst material has an ethylene selectivity of at least 90% at an ethane conversion of at least 45% in an oxidative dehydrogenation reaction of ethane. In some embodiments, the catalyst material has a 45% ethane conversion temperature from 300° C. to 420° C. in an oxidative dehydrogenation reaction of ethane. In some embodiments, the catalyst material has a 45% ethane conversion temperature from 300° C. to 420° C. in an oxidative dehydrogenation reaction of ethane.

The present disclosure also provides a method for preparing a catalyst. The method includes forming a slurry comprising metal oxides, a bismuth compound, a reducing agent, and water; and heating the slurry to form the catalyst. The metal oxides include an oxide of molybdenum; an oxide of vanadium; and an oxide of tantalum or an oxide of niobium, or both. The bismuth compound includes bismuth oxide, bismuth hydroxide, or a bismuth carbonate.

In some embodiments, a ratio of the water in the slurry to amount of catalyst formed is between 0.1 mL water per gram of catalyst and 10 mL water per gram of catalyst. In some embodiments, the ratio of the water in the slurry to amount of catalyst formed is between 0.2 mL water per gram of catalyst and 1 mL water per gram of catalyst.

In some embodiments, the slurry has a ratio of water to metal oxides between 0.2 mL water per gram of metal oxides and 0.6 mL water per gram of metal oxides. In some embodiments, the slurry has a ratio of water to metal oxides between 0.3 mL water per gram of metal oxides and 0.5 mL water per gram of metal oxides.

In some embodiments, the oxide of molybdenum is MoO₃.

In some embodiments, the oxide of vanadium is V₂O₅.

In some embodiments, the oxide of tantalum, when present, is Ta₂O₅·xH₂O, and the oxide of niobium, when present, is Nb₂O₅·xH₂O.

In some embodiments, the bismuth compound is bismuth hydroxide.

In some embodiments, the method further includes grinding, wet milling, dry milling, or crushing the metal oxides and the bismuth compound. In some embodiments, the method further includes grinding, wet milling, dry milling, or crushing the reducing agent.

In some embodiments, the reducing agent includes an alcohol, a carboxylic acid, or an ester. In some embodiments, the reducing agent is oxalic acid. In some embodiments, the slurry includes no more than one reducing agent.

In some embodiments, the method includes heating the slurry by ramping a temperature from ambient to a temperature between 100° C. and 200° C. over a ramping time between 2 hours and 48 hours; and holding the temperature at a holding temperature between 100° C. and 200° C. for a holding time between 12 hours and 160 hours.

In some embodiments, the method includes heating the slurry by ramping a temperature from ambient to a temperature between 100° C. and 200° C. over a ramping time between 2 hours and 48 hours; and holding the temperature at a holding temperature between 100° C. and 200° C. for a holding time between 12 hours and 120 hours.

In some embodiments, the method further includes washing the catalyst with water.

In some embodiments, the method further includes calcining the catalyst to form a calcined catalyst. In some embodiments, the method includes calcining the catalyst by placing the catalyst in a furnace under an oxygen-free environment; ramping a temperature of the furnace from ambient to a temperature between 500° C. and 620° C. over a ramping time between 2 hours and 10 hours; and holding the temperature of the furnace at a holding temperature between 500° C. and 620° C. for a holding time between 1 hour and 10 hours.

In some embodiments, the metal oxides and the bismuth compound each have a particle size in the range of from 0.5 μm to 250 μm.

In some embodiments, a ratio of a total amount of water used to prepare the catalyst is between 0.2 mL water per gram of catalyst and 25 mL water per gram of catalyst.

The present disclosure also provides a process for oxidative dehydrogenation of ethane. The process includes contacting a gaseous feed comprising ethane and oxygen with a catalyst in a reactor to produce an effluent comprising ethylene, wherein the catalyst has the formula MoₐV_{b}Bi_{c}M_{d}Oₓ, wherein M is Ta or Nb, or a mixture thereof, a is 1.0; b is 0.01 to 0.5; c is 0.005 to 0.2; d is 0.005 to 0.1; and x is the number of oxygen atoms necessary to render the catalyst electrically neutral; wherein a, b, c, and d are determined based on the amount of each starting material used to form the catalyst. In some embodiments, the values of a, b, c, and d are also determined by elemental analysis.

In some embodiments, the catalyst has a formula selected from Mo₁V_{0.31}Bi_{0.04}M_{0.05}Oₓ, Mo₁V_{0.31}Bi_{0.05}M_{0.05}Oₓ, Mo₁V_{0.31}Bi_{0.06}M_{0.05}Oₓ, Mo₁V_{0.32}Bi_{0.05}M_{0.05}Oₓ, Mo₁V_{0.32}Bi_{0.04}M_{0.03}Oₓ, Mo₁V_{0.33}Bi_{0.05}M_{0.04}Oₓ, Mo₁V_{0.26}Bi_{0.06}M_{0.03}Oₓ, Mo₁V_{0.33}Bi_{0.06}M_{0.04}Oₓ, and Mo₁V_{0.26}Bi_{0.05}M_{0.05}Oₓ. In some embodiments, the catalyst has a formula selected from Mo₁V_{0.32}Bi_{0.04}Ta_{0.03}Oₓ, Mo₁V_{0.33}Bi_{0.05}Ta_{0.04}Oₓ, Mo₁V_{0.26}Bi_{0.06}Ta_{0.03}Oₓ, Mo₁V_{0.31}Bi_{0.06}Nb_{0.01}Oₓ, Mo₁V_{0.33}Bi_{0.06}Ta_{0.04}Oₓ, and Mo₁V_{0.26}Bi_{0.05}Ta_{0.05}Oₓ.

In some embodiments, the catalyst is comprised in a catalyst material, the catalyst material comprising a catalyst support or carrier. In some embodiments, the catalyst support or carrier is α-alumina.

In some embodiments, the process for the oxidative dehydrogenation of ethane has a 45% ethane conversion temperature from 300° C. to 420° C. In some embodiments, the process for the oxidation dehydrogenation of ethane has a 45% ethane conversion temperature from 350° C. to 400° C.

In some embodiments, the process for the oxidative dehydrogenation of ethane further comprises converting the ethylene to a product. In some embodiments, the product is a polyethylene is selected from very low density polyethylene (VLDPE), low density polyethylene (LDPE), linear low density polyethylene (LLDPE), medium density polyethylene (MDPE), and high density polyethylene (HDPE).

### BRIEF DESCRIPTION OF DRAWINGS

FIG. 1 is a powder X-ray diffraction (PXRD) pattern of comparative sample 1C.

FIG. 2 is a PXRD pattern of example 2E.

FIG. 3 is a PXRD pattern of example 3E.

FIG. 4 is a PXRD pattern of example 4E.

FIG. 5 is an overlay of PXRD patterns of example 5E uncalcined, calcined, and pelleted and sintered.

FIG. 6 is a PXRD pattern of example 6E.

FIG. 7 is a PXRD pattern of example 7E.

FIG. 8 is an overlay of PXRD patterns of example 8E before and after calcination.

FIG. 9 is an overlay of PXRD patterns of example 9E before and after calcination.

FIG. 10 is a PXRD patter of example 11E before calcination.

FIG. 11 shows scanning electron microscopy (SEM) images of example 2E.

FIG. 12 shows SEM images of example 4E.

FIG. 13 shows SEM images of example 5E.

FIG. 14 shows SEM images of example 1IE.

FIG. 15 shows a Barret-Joyner-Halenda (BJH) plot of pore volume for example 9E.

FIG. 16 is a nitrogen physisorption plot of example 9E.

FIG. 17 is a plot of the conversion (%) of ethane for comparative sample 1C and example 4E.

FIG. 18 is a plot of the selectivity of ethylene formation for comparative sample 1C and example 4E.

FIG. 19 is a plot of the long-term ODH performance study for catalyst 5E-pelleted.

### DESCRIPTION OF EMBODIMENTS

Reference will now be made in detail to certain embodiments of the disclosed subject matter, examples of which are illustrated in part in the accompanying figures. While the disclosed subject matter will be described in conjunction with the enumerated claims, it will be understood that the exemplified subject matter is not intended to limit the claims.

Selective oxidation (SO) is generally used in oxidative dehydrogenation (ODH) reactions to form alpha-olefins from corresponding alkanes, such as to form ethylene from ethane. Provided in this disclosure is an oxidative dehydrogenation catalyst that includes molybdenum (Mo); vanadium (V); bismuth (Bi); tantalum (Ta), niobium (Nb), or both; and oxygen (O). The present disclosure also provides a synthesis method for a bismuth-doped catalyst according to the present disclosure, for example, for use in an ODH process.

In the method for preparing a catalyst provided herein, catalyst precursor powders are mixed, for example, by being ground together, and then used in a hydrothermal synthesis process to form the catalyst. Previous ODH catalysts have included a tellurium or an antimony dopant. However, bismuth has lower toxicity than tellurium or antimony. In addition, in embodiments of the catalysts described herein, the bismuth-doped catalysts have a combination of high activity for ethane oxidative dehydrogenation and high ethylene selectivity.

Further, in embodiments of the method for preparing the catalysts described herein, lower amounts of water are used, significantly reducing the amount of heavy metal contaminated waste produced during synthesis.

Further, the methods provided herein do not require complex mixing or milling steps that have been reported in previous synthetic procedures for related MoVNbTe oxides. The methods provided herein offer significant cost savings and simplification over previously reported synthetic procedures.

As used herein, the term “catalyst” generally refers to the active portion of a catalyst material that can promote oxidative dehydrogenation, such as the oxidative dehydrogenation of ethane to ethylene. The catalyst may be processed in further steps to form a catalyst material. The catalyst material may also be processed in further steps to form a final catalyst material.

The catalyst provided herein is represented by the formula MoₐV_{b}Bi_{c}M_{d}Oₓ, wherein M is Ta or Nb, or a mixture thereof. In some embodiments, the catalyst has a formula selected from MoₐV_{b}Bi_{c}Ta_{d}Oₓ, and MoₐV_{b}Bi_{c}Nb_{d}Oₓ. The values of a, b, c, and d may refer to the values based on the amount of each starting material used to form the catalyst, such as the amount of each metal oxide and bismuth compound added to the slurry to prepare the catalyst. The values of a, b, c, and d may also refer to values measured by elemental analysis, for example by inductively coupled plasma mass spectroscopy (ICP-MS), neutron activation analysis (NAA), X-ray fluorescence (XRF), ion chromatography mass spectrometry (IC-MS), proton induced X-ray emission (PIXE), or energy-dispersive X-ray spectroscopy (EDX). In the alternative, if specified, the values a, b, c, and d may only refer to the values determined by elemental analysis, for example by ICP-MS, NAA, XRF, IC-MS, PIXE, or EDX.

The catalyst formula with respect to the ratios of values a, b, c, and d may be selected in order to affect the activity, selectivity, purity, and stability of the catalyst. In some embodiments, the value of d is minimized to lower costs while maintaining good catalyst performance.

In some embodiments, a is 1.0.

In some embodiments, b is 0.01 to 0.5. In some embodiments, b is 0.01 to 0.4. In some embodiments, b is 0.01 to 0.3. In some embodiments, b is 0.1 to 0.5. In some embodiments, b is 0.1 to 0.4. In some embodiments, b is 0.1 to 0.3. In some embodiments, b is 0.2 to 0.5. In some embodiments, b is 0.2 to 0.4. In some embodiments, b is 0.20 to 0.35. In some embodiments, b is 0.25 to 0.35. In some embodiments, b is 0.3 to 0.4. In some embodiments, b is 0.30 to 0.35. In some embodiments, b is 0.3. In some embodiments, b is 0.25. In some embodiments, b is 0.26. In some embodiments, b is 0.27. In some embodiments, b is 0.32. In some embodiments, b is 0.33. In some embodiments, b is 0.34.

In some embodiments, c is 0.005 to 0.2. In some embodiments, c is 0.01 to 0.2. In some embodiments, c is 0.01 to 0.1. In some embodiments, c is 0.01 to 0.09. In some embodiments, c is 0.01 to 0.07. In some embodiments, c is 0.02 to 0.1. In some embodiments, c is 0.02 to 0.09. In some embodiments, c is 0.02 to 0.07. In some embodiments, c is 0.03 to 0.1. In some embodiments, c is 0.03 to 0.09. In some embodiments, c is 0.03 to 0.07. In some embodiments, c is 0.04 to 0.06. In some embodiments, c is 0.06. In some embodiments, c is 0.05. In some embodiments, c is 0.04.

In some embodiments, d is 0.005 to 0.1. In some embodiments, d is 0.01 to 0.10. In some embodiments, d is 0.01 to 0.09. In some embodiments, d is 0.01 to 0.07. In some embodiments, d is 0.01 to 0.05. In some embodiments, d is 0.01 to 0.04. In some embodiments, d is 0.02 to 0.1. In some embodiments, d is 0.02 to 0.09. In some embodiments, d is 0.02 to 0.07. In some embodiments, d is 0.02 to 0.05. In some embodiments, d is 0.02 to 0.04. In some embodiments, d is 0.03 to 0.05. In some embodiments, d is 0.03. In some embodiments, d is 0.04. In some embodiments, d is 0.05.

In each catalyst formulation according to the present disclosure, oxygen is present in sufficient amounts to render the catalyst electrically neutral. The skilled person will appreciate that oxygen-containing species may also be adsorbed or trapped by the catalyst.

In some embodiments, the catalyst has a formula MoₐV_{b}Bi_{c}Ta_{d}Oₓ, wherein the values of a, b, c, and d are as described herein. In some embodiments, the catalyst has a formula MoₐV_{b}Bi_{c}Ta_{d}Oₓ, wherein a is 1.0, b is 0.01 to 0.5, c is 0.005 to 0.2, and d is 0.005 to 0.1. In some embodiments, the catalyst has a formula MoₐV_{b}Bi_{c}Ta_{d}Oₓ, wherein a is 1.0, b is 0.2 to 0.4, c is 0.01 to 0.07, and d is 0.01 to 0.07. In some embodiments, the catalyst has a formula MoₐV_{b}Bi_{c}Ta_{d}Oₓ, wherein a is 1.0, b is 0.2 to 0.4, c is 0.01 to 0.07, and d is 0.01 to 0.05. In some embodiments, the catalyst has a formula MoₐV_{b}Bi_{c}Ta_{d}Oₓ, wherein a is 1.0, b is 0.2 to 0.4, c is 0.04 to 0.06, and d is 0.03 to 0.05. In some embodiments, the catalyst has a formula MoₐV_{b}Bi_{c}Ta_{d}Oₓ, wherein a is 1.0, b is 0.3 to 0.4, c is 0.03 to 0.08, and d is 0.03 to 0.06. In some embodiments, the catalyst has a formula MoₐV_{b}Bi_{c}Ta_{d}Oₓ, wherein a is 1.0, b is 0.2 to 0.4, c is 0.04 to 0.06, and d is 0.02 to 0.05. In some embodiments, the catalyst has a formula MoₐV_{b}Bi_{c}Ta_{d}Oₓ, wherein a is 1.0, b is 0.30 to 0.35; c is 0.04 to 0.06; and d is 0.03 to 0.05.

In some embodiments, the catalyst has a formula MoₐV_{b}Bi_{c}Nb_{d}Oₓ, wherein the values of a, b, c, and d are as described herein. In some embodiments, the catalyst has a formula MoₐV_{b}Bi_{c}Nb_{d}Oₓ, wherein a is 1.0, b is 0.01 to 0.5, c is 0.005 to 0.2, and d is 0.005 to 0.1. In some embodiments, the catalyst has a formula MoₐV_{b}Bi_{c}Nb_{d}Oₓ, wherein a is 1.0, b is 0.2 to 0.4, c is 0.01 to 0.07, and d is 0.01 to 0.07. In some embodiments, the catalyst has a formula MoₐV_{b}Bi_{c}Nb_{d}Oₓ, wherein a is 1.0, b is 0.1 to 0.5, c is 0.01 to 0.1, and d is 0.005 to 0.1. In some embodiments, the catalyst has a formula MoₐV_{b}Bi_{c}Nb_{d}Oₓ, wherein a is 1.0, b is 0.2 to 0.4, c is 0.04 to 0.06, and d is 0.02 to 0.05. In some embodiments, the catalyst has a formula MoₐV_{b}Bi_{c}Nb_{d}Oₓ, wherein a is 1.0, b is 0.30 to 0.40, c is 0.03 to 0.8, and d is 0.03 to 0.06. In some embodiments, the catalyst has a formula MoₐV_{b}Bi_{c}Nb_{d}Oₓ, wherein a is 1.0, b is 0.30 to 0.35; c is 0.04 to 0.05; and d is 0.03 to 0.05.

In some embodiments, the values of a, b, c, and d are determined based on the amount of each starting material used to form the catalyst. For example, the values of a, b, c, and d are determined based on the amount (molar equivalents) of each metal oxide and bismuth compound added to the slurry. In some embodiments, the catalyst has the formula Mo₁V_{0.31}Bi_{0.05}M_{0.05}Oₓ, wherein the formula is determined based on the amount of each starting material used to form the catalyst. In some embodiments, the catalyst has the formula Mo₁V_{0.31}Bi_{0.05}Ta_{0.05}Oₓ, wherein the formula is determined based on the amount of each starting material used to form the catalyst. In some embodiments, the catalyst has the formula Mo₁V_{0.31}Bi_{0.05}Nb_{0.05}Oₓ, wherein the formula is determined based on the amount of each starting material used to form the catalyst.

In some embodiments, the values of a, b, c, and d are determined by elemental analysis, such as by energy-dispersive X-ray spectroscopy (EDX). In some embodiments, the catalyst has a formula selected from Mo₁V_{0.26}Bi_{0.06}Ta_{0.03}Oₓ, Mo₁V_{0.32}Bi_{0.04}Ta_{0.03}Oₓ, Mo₁V_{0.33}Bi_{0.05}Ta_{0.04}Oₓ, Mo₁V_{0.33}Bi_{0.06}Ta_{0.04}Oₓ, and Mo₁V_{0.26}Bi_{0.05}Ta_{0.05}Oₓ, wherein the formula is determined by EDX. In some embodiments, the catalyst has the formula Mo₁V_{0.32}Bi_{0.04}Ta_{0.03}Oₓ, wherein the formula is determined by EDX. In some embodiments, the catalyst has the formula Mo₁V_{0.33}Bi_{0.05}Ta_{0.04}Oₓ, wherein the formula is determined by EDX. In some embodiments, the catalyst has the formula Mo₁V_{0.26}Bi_{0.06}Ta_{0.03}Oₓ, wherein the formula is determined by EDX. In some embodiments, the catalyst has the formula Mo₁V_{0.33}Bi_{0.06}Ta_{0.04}Oₓ, wherein the formula is determined by EDX. In some embodiments, the catalyst has the formula Mo₁V_{0.26}Bi_{0.05}Ta_{0.05}Oₓ, wherein the formula is determined by EDX. In some embodiments, the catalyst has the formula Mo₁V_{0.32}Bi_{0.04}Ta_{0.03}Oₓ, wherein the formula is determined by EDX.

In some embodiments, the catalyst is characterized by having at least five powder X-ray diffraction peaks (2θ degrees) chosen from 6.6°±0.2°, 7.8°±0.2°, 8.9°±0.2°, 10.7°±0.2°, 12.7°±0.2°, 13.9°±0.2°, 22.2°±0.2°, 23.3°±0.2°, 25.1°±0.2°, 25.7°±0.2°, 26.1° ±0.2°, 26.7±0.2°, 27.10°±0.2°, 28.10°±0.2°, 29.10°±0.2°, 31.30°±0.2°, 35.20°±0.2°, 39.0°±0.2°, 45.3°±0.2°, 48.5°±0.2°, 49.5°±0.2°, 51.1°±0.2°, 53.4±0.2°, 54.9°±0.2°, 56.40°±0.2°, 57.60°±0.2°, 62.80°±0.2°, wherein the PXRD pattern is obtained using Cu Kα radiation.

In some embodiments, the catalyst is characterized by having at least five powder X-ray diffraction peaks (2θ degrees) chosen from 6.6°±0.2°, 7.8°±0.2°, 8.9°±0.2°, 10.7°±0.2°, 13.9°±0.2°, 22.2°±0.2°, 25.1°±0.2°, 26.1°±0.2°, 26.7±0.2°, 27.1°±0.2°, 28.1°±0.2° 29.1°±0.2° 31.3°±0.2° 35.2°±0.2° 45.3°±0.2°, 48.5°±0.2°, 49.5°±0.2°, 51.1°±0.2°, 53.4±0.2°, 54.9°±0.2°, 56.4°±0.2°, 57.6°±0.2°, and 62.8°+0.2°, wherein the PXRD pattern is obtained using Cu Kα radiation.

In some embodiments the catalyst is characterized by having at least ten powder X-ray diffraction peaks (2θ degrees) chosen from 6.6°±0.2°, 7.8°±0.2°, 8.9°±0.2°, 10.7°±0.2°, 12.7°±0.2°, 13.9°±0.2°, 22.2°±0.2°, 23.3°±0.2°, 25.1°±0.2°, 25.7°±0.2°, 26.1°±0.2°, 26.7±0.2°, 27.10°±0.2°, 28.10°±0.2°, 29.10°±0.2°, 31.30°±0.2°, 35.20°±0.2°, 39.0°±0.2°, 45.3°±0.2°, 48.5°±0.2°, 49.5°±0.2°, 51.1°±0.2°, 53.4±0.2°, 54.9°±0.2°, 56.40°±0.2°, 57.60°±0.20, and 62.80°±0.2°, wherein the PXRD pattern is obtained using Cu Kα radiation.

In some embodiments the catalyst is characterized by having at least ten powder X-ray diffraction peaks (2θ degrees) chosen from 6.6°±0.2°, 7.8°±0.2°, 8.9°±0.2°, 10.7°±0.2°, 13.9°±0.2°, 22.2°±0.2°, 25.1°±0.2°, 26.1°±0.2°, 26.7±0.2°, 27.1°±0.2°, 28.1°±0.2°, 29.1°±0.2°, 31.3°±0.2°, 35.2°±0.2°, 45.3°±0.2°, 48.5°±0.2°, 49.5°±0.2°, 51.10°±0.2°, 53.4±0.2°, 54.90°±0.2°, 56.40°±0.2°, 57.60°±0.20, and 62.80°±0.2°, wherein the PXRD pattern is obtained using Cu Kα radiation.

In some embodiments the catalyst is characterized by having at least fifteen powder X-ray diffraction peaks (2θ degrees) chosen from 6.6°±0.2°, 7.8°±0.2°, 8.9°±0.2°, 10.7°±0.2°, 12.7°±0.2°, 13.9°±0.2°, 22.2°±0.2°, 23.3°±0.2°, 25.1°±0.2°, 25.7°±0.2°, 26.10°±0.2°, 26.7±0.2°, 27.10°±0.2°, 28.10°±0.2°, 29.10°±0.2°, 31.30°±0.2°, 35.20°±0.2°, 39.0°±0.2°, 45.3°±0.2°, 48.5°±0.2°, 49.5°±0.2°, 51.1°±0.2°, 53.4±0.2°, 54.9°±0.2°, 56.4°±0.2°, 57.6°±0.2°, and 62.8°±0.2°, wherein the PXRD pattern is obtained using Cu Kα radiation.

In some embodiments the catalyst is characterized by having at least fifteen powder X-ray diffraction peaks (2θ degrees) chosen from 6.6°±0.2°, 7.8°±0.2°, 8.9°±0.2°, 10.7°±0.2°, 13.9°±0.2°, 22.2°±0.2°, 25.1°±0.2°, 26.1°±0.2°, 26.7±0.2°, 27.1°±0.2°, 28.10°±0.2°, 29.10°±0.2°, 31.30°±0.2°, 35.20°±0.2°, 45.30°±0.2°, 48.50°±0.2°, 49.50°±0.2°, 51.1°±0.2°, 53.4±0.2°, 54.9°±0.2°, 56.4°±0.2°, 57.6°±0.20, and 62.8°±0.20, wherein the PXRD pattern is obtained using Cu Kα radiation.

In some embodiments the catalyst is characterized by having at least five powder X-ray diffraction peaks (2θ degrees) chosen from 7.8±0.2°, 22.2±0.2°, 26.7±0.2°, 27.1±0.2°, 29.1±0.2°, 35.2±0.2°, 45.3±0.2°, and 48.5±0.2°, wherein the PXRD pattern is obtained using Cu Kα radiation.

In some embodiments the catalyst is characterized by having powder X-ray diffraction peaks (2θ degrees) at 7.8±0.2°, 22.2±0.2°, 27.1±0.2°, 35.2±0.2°, and 45.3±0.2°, wherein the PXRD pattern is obtained using Cu Kα radiation.

In some embodiments, the PXRD includes peaks (2θ degrees) corresponding to MoO₃, which may be unreacted MoO₃.

In some embodiments, the catalyst has a Brunauer-Emmett-Teller (BET) surface area as determined by nitrogen physisorption analysis between 5 m²/g and 60 m²/g, between 5 m²/g and 50 m²/g, between 5 m²/g and 40 m²/g, between 5 m²/g and 30 m²/g, between 5 m²/g and 25 m²/g, between 5 m²/g and 20 m²/g, between 5 m²/g and 15 m²/g, or between 5 m²/g and 10 m²/g. In some embodiments, the catalyst has a BET surface area as determined by nitrogen physisorption analysis between 10 m²/g and 40 m²/g, between 10 m²/g and 30 m²/g, between 10 m²/g and 25 m²/g, or between 10 m²/g and 20 m²/g. In some embodiments, the catalyst is calcined and has a BET surface area as determined by nitrogen physisorption analysis of 10 m²/g, 11 m²/g, 12 m²/g, 13 m²/g, 14 m²/g, 15 m²/g, 16 m²/g, 17 m²/g, 18 m²/g, 19 m²/g, or 20 m²/g. In some embodiments, the catalyst is uncalcined and has a BET surface area as determined by nitrogen physisorption analysis of 50 m²/g, 51 m²/g, 52 m²/g, 53 m²/g, 54 m²/g, 55 m²/g, 56 m²/g, 57 m²/g, 58 m²/g, 59 m²/g, or 60 m²/g.

In some embodiments, the catalyst has a pore volume as determined by nitrogen physisorption analysis with a Barrett-Joyner-Halenda (BJH) model between 0.02 cm³/g and 0.25 cm³/g. In some embodiments, the catalyst has a pore volume as determined by nitrogen physisorption analysis with a BJH model between 0.02 and 0.2 cm³/g. In some embodiments, the catalyst has a pore volume as determined by nitrogen physisorption analysis with a BJH model between 0.05 cm³/g and 0.2 cm³/g, between 0.05 cm³/g and 0.15 cm³/g, or between 0.05 cm³/g and 0.1 cm³/g. In some embodiments, the catalyst has a pore volume as determined by nitrogen physisorption analysis with a BJH model of 0.02 cm³/g, 0.03 cm³/g, 0.04 cm³/g, 0.05 cm³/g, 0.0.6 cm³/g, 0.07 cm³/g, 0.08 cm³/g, 0.09 cm³/g, 0.1 cm³/g, 0.11 cm³/g, 0.12 cm³/g, 0.13 cm³/g, 0.14 cm³/g, 0.15 cm³/g, 0.16 cm³/, 0.17 cm³/g, 0.18 cm³/g, 0.19 cm³/g, or 0.2 cm³/g.

In some embodiments, the catalyst has a formula of MoₐV_{b}Bi_{c}Ta_{d}Oₓ, wherein a is 1.0, b 0.01 to 0.5, c is 0.005 to 0.2, d is 0.005 to 0.1, and x is the number of oxygen atoms necessary to render the catalyst electrically neutral; a pore volume between 0.02 cm³/g and 0.25 cm³/g as determined by nitrogen physisorption analysis with a Barrett-Joyner-Halenda model; a Brunauer-Emmett-Teller surface area between 5 m²/g and 60 m²/g as determined by nitrogen physisorption analysis; and at least five powder X-ray diffraction (PXRD) peaks (2θ degrees) chosen from 6.6°±0.2°, 7.8°±0.2°, 8.9°±0.2°, 10.7°±0.2°, 12.7°±0.2°, 13.9°±0.2°, 22.2°±0.2°, 23.3°±0.2°, 25.1°±0.2°, 25.7°±0.2°, 26.1°±0.2°, 26.7±0.2°, 27.10°±0.2°, 28.10°±0.2°, 29.10°±0.2°, 31.30°±0.2°, 35.20°±0.2°, 39.00°±0.2°, 45.30° 0.2°, 48.5°±0.2°, 49.5°±0.2°, 51.1°±0.2°, 53.4±0.2°, 54.9°±0.2°, 56.4°±0.2°, 57.6° 0.2°, 62.8°±0.2°, wherein the PXRD pattern is obtained using Cu Kα radiation.

In some embodiments, the catalyst has a formula of MoₐV_{b}Bi_{c}Ta_{d}Oₓ, wherein a is 1.0, b 0.01 to 0.5, c is 0.005 to 0.2, d is 0.005 to 0.1, wherein a, b, c and d are determined by energy-dispersive X-ray spectroscopy, and x is the number of oxygen atoms necessary to render the catalyst electrically neutral; a pore volume between 0.02 cm³/g and 0.25 cm³/g as determined by nitrogen physisorption analysis with a Barrett-Joyner-Halenda model; a Brunauer-Emmett-Teller surface area between 5 m²/g and 60 m²/g as determined by nitrogen physisorption analysis; and at least five powder X-ray diffraction (PXRD) peaks (2θ degrees) chosen from 6.6°±0.2°, 7.8°±0.2°, 8.9°±0.2°, 10.7°±0.2°, 12.7°±0.2°, 13.9°±0.2°, 22.2°±0.2°, 23.3°±0.2°, 25.1°±0.2°, 25.7°±0.2°, 26.1°±0.2°, 26.7±0.2°, 27.10°±0.2°, 28.10°±0.2°, 29.10°±0.2°, 31.30°±0.2°, 35.20°±0.2°, 39.00°±0.2°, 45.30° 0.2°, 48.5°±0.2°, 49.5°±0.2°, 51.1°±0.2°, 53.4±0.2°, 54.9°±0.2°, 56.4°±0.2°, 57.6°±0.2°, 62.8°±0.2°, wherein the PXRD pattern is obtained using Cu Kα radiation.

In some embodiments, the catalyst has a formula of MoₐV_{b}Bi_{c}Nb_{d}Oₓ, wherein a is 1.0, b 0.01 to 0.5, c is 0.005 to 0.2, d is 0.005 to 0.1, and x is the number of oxygen atoms necessary to render the catalyst electrically neutral; a pore volume between 0.02 cm³/g and 0.25 cm³/g as determined by nitrogen physisorption analysis with a Barrett-Joyner-Halenda model; a Brunauer-Emmett-Teller surface area between 5 m²/g and 60 m²/g as determined by nitrogen physisorption analysis; and at least five powder X-ray diffraction (PXRD) peaks (2θ degrees) chosen from 6.6°±0.2°, 7.8°±0.2°, 8.9°±0.2°, 10.7°±0.2°, 12.7°±0.2°, 13.9°±0.2°, 22.2°±0.2°, 23.3°±0.2°, 25.1°±0.2°, 25.7°±0.2°, 26.1°±0.2°, 26.7±0.2°, 27.10°±0.2°, 28.10°±0.2°, 29.10°±0.2°, 31.30°±0.2°, 35.20°±0.2°, 39.00°±0.2°, 45.30° 0.2°, 48.5°±0.2°, 49.5°±0.2°, 51.1°±0.2°, 53.4±0.2°, 54.9°±0.2°, 56.4°±0.2°, 57.6°±0.2°, 62.8°±0.2°, wherein the PXRD pattern is obtained using Cu Kα radiation.

In some embodiments, the catalyst has a formula of MoₐV_{b}Bi_{c}Nb_{d}Oₓ, wherein a is 1.0, b 0.01 to 0.5, c is 0.005 to 0.2, d is 0.005 to 0.1, wherein a, b, c and d are determined by energy-dispersive X-ray spectroscopy, and x is the number of oxygen atoms necessary to render the catalyst electrically neutral; a pore volume between 0.02 cm³/g and 0.25 cm³/g as determined by nitrogen physisorption analysis with a Barrett-Joyner-Halenda model; a Brunauer-Emmett-Teller surface area between 5 m²/g and 60 m²/g as determined by nitrogen physisorption analysis; and at least five powder X-ray diffraction (PXRD) peaks (2θ degrees) chosen from 6.6°±0.2°, 7.8°±0.2°, 8.9°±0.2°, 10.7°±0.2°, 12.7°±0.2°, 13.9°±0.2°, 22.2°±0.2°, 23.3° 0 0.2°, 25.1°±0.2, 25.70°±0.2, 26.1°±0.2°, 26.7±0.2°, 27.10°±0.2°, 28.10°±0.2°, 29.10°±0.2°, 31.30°±0.2°, 35.20°±0.2°, 39.00°±0.2°, 45.30° 0.2°, 48.5°±0.2°, 49.5°±0.2°, 51.1°±0.2°, 53.4±0.2°, 54.9°±0.2°, 56.4°±0.2°, 57.6°±0.2°, 62.8°±0.2°, wherein the PXRD pattern is obtained using Cu Kα radiation.

In some embodiments, the catalyst has a formula of MoₐV_{b}Bi_{c}Ta_{d}Oₓ, wherein a is 1.0, b 0.3 to 0.4, c is 0.05 to 0.7, d is 0.03 to 0.05, and x is the number of oxygen atoms necessary to render the catalyst electrically neutral; a pore volume between 0.1 cm³/g and 0.2 cm³/g as determined by nitrogen physisorption analysis with a Barrett-Joyner-Halenda model; a Brunauer-Emmett-Teller surface area between 10 m²/g and 25 m²/g as determined by nitrogen physisorption analysis; and at least five powder X-ray diffraction (PXRD) peaks (2θ degrees) chosen from 6.6°±0.2°, 7.8°±0.2°, 8.9°±0.2°, 10.7°±0.2°, 12.7°±0.2°, 13.9°±0.2°, 22.2°±0.2°, 23.3°±0.2°, 25.1°±0.2°, 25.7°±0.2°, 26.1°±0.2°, 26.7±0.2°, 27.10°±0.2°, 28.10°±0.2°, 29.10°±0.2°, 31.30°±0.2°, 35.20°±0.2°, 39.00°±0.2°, 45.30° 0.2°, 48.5°±0.2°, 49.5°±0.2°, 51.1°±0.2°, 53.4±0.2°, 54.9°±0.2°, 56.4°±0.2°, 57.6° 0.2°, 62.8°±0.2°, wherein the PXRD pattern is obtained using Cu Kα radiation.

In some embodiments, the catalyst has a formula of MoₐV_{b}Bi_{c}Nb_{d}Oₓ, wherein a is 1.0, b 0.3 to 0.4, c is 0.05 to 0.7, d is 0.03 to 0.05, and x is the number of oxygen atoms necessary to render the catalyst electrically neutral; a pore volume between 0.1 cm³/g and 0.2 cm³/g as determined by nitrogen physisorption analysis with a Barrett-Joyner-Halenda model; a Brunauer-Emmett-Teller surface area between 10 m²/g and 25 m²/g as determined by nitrogen physisorption analysis; and at least five powder X-ray diffraction (PXRD) peaks (2θ degrees) chosen from 6.6°±0.2°, 7.8°±0.2°, 8.9°±0.2°, 10.7°±0.2°, 12.7°±0.2°, 13.9°±0.2°, 22.2°±0.2°, 23.3°±0.2°, 25.1°±0.2°, 25.7°±0.2°, 26.1°±0.2°, 26.7±0.2°, 27.10°±0.2°, 28.10°±0.2°, 29.10°±0.2°, 31.30°±0.2°, 35.20°±0.2°, 39.00°±0.2°, 45.30° 0.2°, 48.5°±0.2°, 49.5°±0.2°, 51.1°±0.2°, 53.4±0.2°, 54.9°±0.2°, 56.4°±0.2°, 57.6° 0.2°, 62.8°±0.2°, wherein the PXRD pattern is obtained using Cu Kα radiation.

In some embodiments, the catalyst has a formula selected from Mo₁V_{0.32}Bi_{0.04}Ta_{0.03}Oₓ, Mo₁V_{0.33}Bi_{0.05}Ta_{0.04}Oₓ, Mo₁V_{0.26}Bi_{0.06}Ta_{0.03}Oₓ, Mo₁V_{0.31}Bi_{0.06}Nb_{0.01}Oₓ, and Mo₁V_{0.33}Bi_{0.06}Ta_{0.04}Oₓ, wherein x is the number of oxygen atoms necessary to render the catalyst electrically neutral; a pore volume between 0.1 cm³/g and 0.2 cm³/g as determined by nitrogen physisorption analysis with a Barrett-Joyner-Halenda model; a Brunauer-Emmett-Teller surface area between 10 m²/g and 25 m²/g as determined by nitrogen physisorption analysis; and at least five powder X-ray diffraction (PXRD) peaks (2θ degrees) chosen from 6.6°±0.2°, 7.8°±0.2°, 8.9°±0.2°, 10.7°±0.2°, 12.7°±0.2°, 13.9°±0.2°, 22.2°±0.2°, 23.3°±0.2°, 25.1°±0.2°, 25.7°±0.2°, 26.1°±0.2°, 26.7±0.2°, 27.1°±0.2°, 28.1°±0.2°, 29.1°±0.2°, 31.3°±0.2°, 35.2°±0.2°, 39.0°±0.2°, 45.3°±0.2°, 48.5°±0.2°, 49.5°±0.2°, 51.1°±0.2°, 53.4±0.2°, 54.9°±0.2°, 56.4°±0.2°, 57.6°±0.2°, and 62.8°±0.2°, wherein the PXRD pattern is obtained using Cu Kα radiation.

In some embodiments, the catalyst has a formula selected from Mo₁V_{0.32}Bi_{0.04}Ta_{0.03}Oₓ, Mo₁V_{0.33}Bi_{0.05}Ta_{0.04}Oₓ, Mo₁V_{0.26}Bi_{0.06}Nb_{0.03}Oₓ, Mo₁V_{0.31}Bi_{0.06}Nb_{0.01}Oₓ, and Mo₁V_{0.33}Bi_{0.06}Ta_{0.04}Oₓ, wherein x is the number of oxygen atoms necessary to render the catalyst electrically neutral; a pore volume between 0.1 cm³/g and 0.2 cm³/g as determined by nitrogen physisorption analysis with a Barrett-Joyner-Halenda model; a Brunauer-Emmett-Teller surface area between 10 m²/g and 25 m²/g as determined by nitrogen physisorption analysis; and at least ten powder X-ray diffraction (PXRD) peaks (2θ degrees) chosen from 6.6°±0.2°, 7.8°±0.2°, 8.9°±0.2°, 10.7°±0.2°, 12.7°±0.2°, 13.9°±0.2°, 22.2°±0.2°, 23.3°±0.2°, 25.1°±0.2°, 25.7°±0.2°, 26.1°±0.2°, 26.7±0.2°, 27.1°±0.2°, 28.1°±0.2°, 29.1°±0.2°, 31.3°±0.2°, 35.2°±0.2°, 39.0°±0.2°, 45.3°±0.2°, 48.5°±0.2°, 49.5°±0.2°, 51.1°±0.2°, 53.4±0.2°, 54.9°±0.2°, 56.4°±0.2°, 57.6°±0.2°, and 62.8°±0.2°, wherein the PXRD pattern is obtained using Cu Kα radiation.

In some embodiments, the catalyst has a formula selected from Mo₁V_{0.32}Bi_{0.04}Ta_{0.03}Oₓ, Mo₁V_{0.33}Bi_{0.05}Ta_{0.04}Oₓ, Mo₁V_{0.26}Bi_{0.06}Nb_{0.03}Oₓ, Mo₁V_{0.31}Bi_{0.06}Nb_{0.01}Oₓ, and Mo₁V_{0.33}Bi_{0.06}Ta_{0.04}Oₓ, wherein x is the number of oxygen atoms necessary to render the catalyst electrically neutral; a pore volume between 0.1 cm³/g and 0.15 cm³/g as determined by nitrogen physisorption analysis with a Barrett-Joyner-Halenda model; a Brunauer-Emmett-Teller surface area between 15 m²/g and 18 m²/g as determined by nitrogen physisorption analysis; and at least five powder X-ray diffraction (PXRD) peaks (2θ degrees) chosen from 6.6°±0.2°, 7.8°±0.2°, 8.9°±0.2°, 10.7°±0.2°, 12.7°±0.2°, 13.9°±0.2°, 22.2°±0.2°, 23.3°±0.2°, 25.1°±0.2°, 25.7°±0.2°, 26.1°±0.2°, 26.7±0.2°, 27.1°±0.2°, 28.1°±0.2°, 29.1°±0.2°, 31.3°±0.2°, 35.2°±0.2°, 39.0°±0.2°, 45.3°±0.2°, 48.5°±0.2°, 49.5°±0.2°, 51.1°±0.2°, 53.4±0.2°, 54.9°±0.2°, 56.4°±0.2°, 57.6°±0.2°, and 62.8°±0.2°, wherein the PXRD pattern is obtained using Cu Kα radiation.

Also provided herein is a catalyst material that includes a catalyst, such as a catalyst of the present disclosure, and a catalyst support or carrier. As used herein, the term “catalyst material” refers to a material that includes an active catalyst that can promote the oxidative dehydrogenation of ethane to ethylene. The catalyst material may be substantially comprised of the catalyst. The catalyst material can be a plurality of particles or a formed catalyst material. Non-limiting examples of formed catalyst materials include extruded catalyst materials, 3D-printed catalyst materials, spheronized catalyst materials, pressed catalyst materials, and cast catalyst materials. Non-limiting examples of pressed and cast catalyst materials include pellets-such as tablets, ovals, and spherical particles. Binder may be used to aid in forming the catalyst material. Catalyst material formation may also include optional workup steps such as: debinding, calcining/sintering, and/or activating/pre-treatment. Workup steps may be introduced to prepare the catalyst material to be loaded into a reactor and produce an expected productivity and mitigate any unexpected thermal runaways during startup.

Some carriers or supports are particularly suitable for the catalyst material, for example, they are chemically compatible (for example, there is no substantial impact on ethylene selectivity or there is an improvement to ethylene selectivity). Other supports may be less compatible, meaning they may lead to substantial reduction of catalyst performance, for example, ethylene selectivity. Consequently, not just any support can be chosen; the support should be selected in a judicious matter based off both short-term and longer-term catalysis performance testing. In some embodiments, there is an emphasis on long-term testing showing no loss of selectivity with time on stream (for example, TOS of >48 hours). As used herein, “time on steam (TOS)” refers to the time the catalyst material spends in the ODH process without interruption.

In some embodiments, the catalyst support or carrier is at least one of precipitated synthetic silica, fumed synthetic silica, silica-alumina, α-alumina, γ-alumina, titania, WO₃—ZrO₂, silicon carbide, MgAl spinel, calcium aluminate, zirconia, and boron nitride. In some embodiments, the catalyst support or carrier is selected from the group consisting of precipitated synthetic silica, fumed synthetic silica, silica-alumina, α-alumina, and anatase titania. In some embodiments, the catalyst support or carrier is α-alumina.

In some embodiments, the catalyst material includes 1 wt. % to 80 wt. %, 10 wt. % to 70 wt. %, 20 wt. % to 60 wt. %, 30 wt. % to 60 wt. %, or 40 wt. % to 60 wt. % of the catalyst support or the carrier. In some embodiments, the catalyst material includes 45 wt. % to 55 wt. %, or 50 wt. % of the catalyst support or carrier.

In some embodiments, the catalyst material includes a lubricant. Non-limiting examples of suitable lubricants include graphite, hexagonal boron nitride, calcium carbonate, a fatty acid, a fatty acid salt, methyl cellulose, polyvinyl alcohol, calcium carbonate, a stearate (for example, magnesium stearate), polyethylene glycol (PEG), glycerol, propylene glycol, or any combination thereof. In some embodiments, the lubricant includes graphite, hexagonal boron nitride, calcium carbonate, a fatty acid, a fatty acid salt, or any combination thereof. In some embodiments, the lubricant includes graphite.

In some embodiments, the catalyst material includes binder. Non-limiting examples of suitable binders include liquid binders, organic binders, inorganic binders, or combinations thereof. Examples of liquid binders include but are not limited to water, oil, sodium silicate, and a polybutadiene emulsion. Examples of organic binders include but are not limited to starch, lignosulfonate, cellulose, cellulose-derived powders (for example, PERIDUR®), microcrystalline cellulose powder (for example, AVICEL®), polyethylene glycol, polyvinyl acetate, polyvinyl alcohol (for example, MOWIOL® 8-88), poly(acrylic acid), other synthetic polymers (for example, ALCOTAC®), and a modified-starch brewery byproduct (for example, Brewex). In some embodiments, the starch is corn starch.

Examples of inorganic binders include but are not limited to bentonite, cement, clay and lime, sodium silicate, calcium aluminate, calcium silicate composite powder, alumina silicate, Fuller's earth, and fly ash chemically activated with alkaline materials.

In some embodiments, the binder includes one or more of a liquid binder, an organic binder, and an inorganic binder. In some embodiments, the binder includes water and at least one of a binder other than water. In some embodiments, the binder includes polyethylene glycol, poly(acrylic acid), and polyvinyl alcohol. In some embodiments, the catalyst material includes 0.1 wt. % to 30 wt. %, 0.5 wt. % to 20 wt. %, or 1 wt. % to 10 wt. % of one or more binders.

In some embodiments, the catalyst material has an axial crush strength between 50 N and 195 N as measured using ASTM D4149-22. In some embodiments, the catalyst material has an axial crush strength between 100 N and 170 N as measured using ASTM D4149-22. In some embodiments, the catalyst material has an axial crush strength between 160 N and 170 N as measured using ASTM D4149-22.

In some embodiments, the catalyst material has a radial crush strength between 80 N and 120 N as measured using ASTM D4149-22. In some embodiments, the catalyst material has a radial crush strength between 90 N and 110 N as measured using ASTM D4149-22. In some embodiments, the catalyst material has a radial crush strength between 95 N and 105 N as measured using ASTM D4149-22.

In some embodiments, the catalyst material has a bulk density between 1.2 g/cm³ and 1.7 g/cm³ as measured using ASTM D3766. In some embodiments the catalyst has a bulk density between 1.4 g/cm³ and 1.6 g/cm³ as measured using ASTM D3766. In some embodiments, the catalyst material has a bulk density between 1.500 g/cm³ and 1.570 g/cm³ as measured using ASTM D3766.

In some embodiments, the catalyst material has a Brunauer-Emmett-Teller (BET) surface area as determined by nitrogen physisorption analysis between 2 m²/g and 10 m²/g, between 3 m²/g and 10 m²/g, between 4 m²/g and 8 m²/g, between 5 m²/g and 8 m²/g, or between 5 m²/g and 6 m²/g. In some embodiments, the catalyst material has a Brunauer-Emmett-Teller (BET) surface area as determined by nitrogen physisorption analysis of 5 m²/g.

In some embodiments, the catalyst material has a pore volume as determined by nitrogen physisorption analysis with a Barrett-Joyner-Halenda (BJH) model of from 0.01 cm³/g to 0.25 cm³/g. In some embodiments, the catalyst material has a pore volume as determined by nitrogen physisorption analysis with a BJH model from 0.02 to 0.2 cm³/g. In some embodiments, the catalyst material has a pore volume as determined by nitrogen physisorption analysis with a BJH model from 0.05 cm³/g to 0.2 cm³/g, from 0.05 cm³/g to 0.15 cm³/g, or from 0.05 cm³/g to 0.1 cm³/g. In some embodiments, the catalyst has a pore volume as determined by nitrogen physisorption analysis with a BJH model of 0.02 cm³/g, 0.03 cm³/g, 0.04 cm³/g, 0.05 cm³/g, 0.0.6 cm³/g, 0.07 cm³/g, 0.08 cm³/g, 0.09 cm³/g, or 0.10 cm³/g.

In some embodiments, the catalyst material is a pellet having a BET surface area of 5 m²/g and a pore volume of 0.02 cm³/g, indicative of a non-porous pellet.

In some embodiments, the catalyst material has a drop strength of at least 80% pellets staying intact when measured using ASTM D8353-20. In some embodiments, the catalyst material has a drop strength of at least 85% pellets staying intact when measured by ASTM D8353-20. In some embodiments, the catalyst material has a drop strength of at least 90% pellets staying intact when measured using ASTM D8353-20. In some embodiments, the catalyst material has a drop strength of at least 95% pellets staying intact when measured using ASTM D8353-20.

In some embodiments, the catalyst material includes a carrier or support, and a catalyst having a formula of MoₐV_{b}Bi_{c}Ta_{d}Oₓ, wherein a is 1.0, b 0.01 to 0.5, c is 0.005 to 0.2, d is 0.005 to 0.1, and x is the number of oxygen atoms necessary to render the catalyst electrically neutral; and the catalyst material has an axial crush strength between 50 N and 195 N as measured using ASTM D4149-22; a radial crush strength between 80 N and 120 N as measured using ASTM D4149-22; and a drop strength of at least 80% pellets staying intact when measured using ASTM D8353-20.

In some embodiments, the catalyst material includes a carrier or support, and a catalyst having a formula of MoₐV_{b}Bi_{c}Ta_{d}Oₓ, wherein a is 1.0, b 0.01 to 0.5, c is 0.005 to 0.2, d is 0.005 to 0.1, and x is the number of oxygen atoms necessary to render the catalyst electrically neutral; the catalyst material has a pore volume between 0.01 cm³/g and 0.25 cm³/g as determined by nitrogen physisorption analysis with a Barrett-Joyner-Halenda model; a Brunauer-Emmett-Teller surface area between 2 m²/g and 10 m²/g as determined by nitrogen physisorption analysis; and a bulk density between 1.2 g/cm³ and 1.7 g/cm³ as measured using ASTM D3766.

In some embodiments, the catalyst material includes a carrier or support, and a catalyst having a formula of MoₐV_{b}Bi_{c}Ta_{d}Oₓ, wherein a is 1.0, b 0.01 to 0.5, c is 0.005 to 0.2, d is 0.005 to 0.1, and x is the number of oxygen atoms necessary to render the catalyst electrically neutral; the catalyst material has an axial crush strength between 50 N and 195 N as measured using ASTM D4149-22; a radial crush strength between 80 N and 120 N as measured using ASTM D4149-22; a pore volume between 0.01 cm³/g and 0.25 cm³/g as determined by nitrogen physisorption analysis with a Barrett-Joyner-Halenda model; a Brunauer-Emmett-Teller surface area between 2 m²/g and 10 m²/g as determined by nitrogen physisorption analysis; a bulk density between 1.2 g/cm³ and 1.7 g/cm³ as measured using ASTM D3766; and a drop strength of at least 80% pellets staying intact when measured using ASTM D8353-20.

In some embodiments, the catalyst material includes a carrier or support, and a catalyst having a formula of MoₐV_{b}Bi_{c}Nb_{d}Oₓ, wherein a is 1.0, b 0.01 to 0.5, c is 0.005 to 0.2, d is 0.005 to 0.1, and x is the number of oxygen atoms necessary to render the catalyst electrically neutral; and the catalyst material has an axial crush strength between 50 N and 195 N as measured using ASTM D4149-22; a radial crush strength between 80 N and 120 N as measured using ASTM D4149-22; and a drop strength of at least 80% pellets staying intact when measured using ASTM D8353-20.

In some embodiments, the catalyst material includes a carrier or support, and a catalyst having a formula of MoₐV_{b}Bi_{c}Nb_{d}Oₓ, wherein a is 1.0, b 0.01 to 0.5, c is 0.005 to 0.2, d is 0.005 to 0.1, and x is the number of oxygen atoms necessary to render the catalyst electrically neutral; the catalyst material has a pore volume between 0.01 cm³/g and 0.25 cm³/g as determined by nitrogen physisorption analysis with a Barrett-Joyner-Halenda model; a Brunauer-Emmett-Teller surface area between 2 m²/g and 10 m²/g as determined by nitrogen physisorption analysis; and a bulk density between 1.2 g/cm³ and 1.7 g/cm³ as measured using ASTM D3766.

In some embodiments, the catalyst material includes a carrier or support, and a catalyst having a formula of MoₐV_{b}Bi_{c}Nb_{d}Oₓ, wherein a is 1.0, b 0.01 to 0.5, c is 0.005 to 0.2, d is 0.005 to 0.1, and x is the number of oxygen atoms necessary to render the catalyst electrically neutral; the catalyst material has an axial crush strength between 50 N and 195 N as measured using ASTM D4149-22; a radial crush strength between 80 N and 120 N as measured using ASTM D4149-22; a pore volume between 0.01 cm³/g and 0.25 cm³/g as determined by nitrogen physisorption analysis with a Barrett-Joyner-Halenda model; a Brunauer-Emmett-Teller surface area between 2 m²/g and 10 m²/g as determined by nitrogen physisorption analysis; a bulk density between 1.2 g/cm³ and 1.7 g/cm³ as measured using ASTM D3766; and a drop strength of at least 80% pellets staying intact when measured using ASTM D8353-20.

Also provided herein is a method for preparing a catalyst including forming a slurry including metal oxides, a bismuth compound, a reducing agent, and water; and heating the slurry to form the catalyst. The metal oxides include an oxide of molybdenum; an oxide of vanadium; and an oxide of tantalum or an oxide of niobium, or both. In some embodiments, the metal oxides include an oxide of molybdenum, an oxide of vanadium, and an oxide of tantalum. In some embodiments, the metal oxides include an oxide of molybdenum, an oxide of vanadium, and an oxide of niobium. In some embodiments, the oxide of molybdenum is MoO₃. In some embodiments, the oxide of vanadium is V₂O₅. In some embodiments, the oxide of tantalum, when present, is Ta₂O₅·xH₂O, and the oxide of niobium, when present, is Nb₂O₅ xH₂O.

The bismuth compound includes bismuth oxide, bismuth hydroxide, or a bismuth carbonate. As used herein, the term “bismuth carbonate” includes basic carbonates of bismuth and oxide-carbonates (subcarbonates) of bismuth. Examples of a bismuth carbonate include bismuth carbonate basic or bismuth subcarbonate ((BiO)₂CO₃). In some embodiments, the bismuth compound is bismuth hydroxide.

The method for preparing a catalyst disclosed herein includes forming a slurry including metal oxides, a bismuth compound, a reducing agent, and water. As used herein, the term “slurry” refers to a mixture of solids in a liquid, and includes a suspension, a paste (that is, the mixture is viscous such that it cannot freely move), or a colloidal solution.

In some embodiments, a ratio of water in the slurry to amount of catalyst formed is between 0.1 mL water per gram of catalyst and 10 mL water per gram of catalyst. As used herein, the phrase “water in the slurry” refers to the amount of water used to form the slurry for the hydrothermal synthesis reaction and does not include water that is not consumed or contaminated during the reaction or water that is used after the reaction. For example, “water in the slurry” does not include water present in the hydrothermal synthesis vessel for heat transfer and/or to maintain a humid atmosphere, or water that is used to wash the catalyst.

As used herein, “water” may refer to deionized water, distilled water, and the like. In some embodiments, the water is distilled water. In some embodiments, the water is distilled, deionized water. In some embodiments, the water may include higher levels of contaminants without harming the catalyst.

In some embodiments, the ratio of water in the slurry to amount of catalyst formed is between 0.1 mL water per gram of catalyst and 5 mL water per gram of catalyst, such as between 0.1 mL water per gram of catalyst and 4 mL water per gram of catalyst, between 0.1 mL water per gram of catalyst and 3 mL water per gram of catalyst, between 0.1 mL water per gram of catalyst and 2 mL water per gram of catalyst, between 0.1 mL water per gram of catalyst and 1 mL water per gram of catalyst, or between 0.1 mL water per gram of catalyst and 0.5 mL water per gram of catalyst. In some embodiments, the ratio of water in the slurry to amount of catalyst formed is between 0.2 mL water per gram of catalyst and 1 mL water per gram of catalyst. In some embodiments, the ratio of water in the slurry to amount of catalyst formed is between 0.3 mL water per gram of catalyst to 0.5 mL water per gram of catalyst.

In some embodiments, the ratio of water in the slurry to the amount of catalyst formed is 0.1 mL water per gram of catalyst, 0.2 mL water per gram of catalyst, 0.3 mL water per gram of catalyst, 0.4 mL water per gram of catalyst, 0.5 mL water per gram of catalyst, 0.6 mL water per gram of catalyst, 0.7 mL water per gram of catalyst, 0.8 mL water per gram of catalyst, 0.9 mL water per gram of catalyst, 1 mL water per gram of catalyst, 2 mL water per gram of catalyst, 3 mL water per gram of catalyst, 4 mL water per gram of catalyst, 5 mL water per gram of catalyst.

In some embodiments, the ratio of water in the slurry to the amount of catalyst formed is 0.25 mL water per gram of catalyst, 0.26 mL water per gram of catalyst, 0.27 mL water per gram of catalyst, 0.28 mL water per gram of catalyst, 0.29 mL water per gram of catalyst, 0.30 mL water per gram of catalyst, 0.31 mL water per gram of catalyst, 0.32 mL water per gram of catalyst, 0.33 mL water per gram of catalyst, 0.34 mL water per gram of catalyst, 0.35 mL water per gram of catalyst, 0.36 mL water per gram of catalyst, 0.37 mL water per gram of catalyst, 0.38 mL water per gram of catalyst, 0.39 mL water per gram of catalyst, or 0.40 mL water per gram of catalyst.

In some embodiments, an optimal ratio of water in the slurry to amount of catalyst formed minimizes the amount of water used in the slurry, and therefore minimizes the amount of wastewater produced, while maintaining saturated vapor pressure in the hydrothermal synthesis reaction.

In some embodiments, the slurry has a ratio of water to metal oxides between 0.1 mL water per gram of metal oxides and 10 mL water per gram of metal oxides, such as between 0.1 mL water per gram of metal oxides and 5 mL water per gram of metal oxides, between 0.1 mL water per gram of metal oxides and 4 mL water per gram of metal oxides, between 0.1 mL water per gram of metal oxides and 3 mL water per gram of metal oxides, between 0.1 mL water per gram of metal oxides and 2 mL water per gram of metal oxides, or between 0.1 mL water per gram of metal oxides and 1 mL water per gram of metal oxides.

The phrase “ratio of water to metal oxides” as used herein refers to the ratio of water used in the slurry to the total mass of metal oxides used in the slurry, which includes an oxide of molybdenum; an oxide of vanadium; and one or both of an oxide of tantalum and an oxide of niobium. In embodiments where the reducing agent is a metal oxide, the total mass of metal oxides used in the slurry does not include the mass of the metal oxide being used as the reducing agent. For example, a slurry comprising 2.7 mL of water, 5.3673 g of MoO₃, 1.0419 g V₂O₅, and 0.4255 of Ta₂O₅·xH₂O would comprise 6.8347 g of total metal oxides and provide a ratio of water to metal oxides of 0.395.

In some embodiments, the slurry has a ratio of water to metal oxides between 0.2 mL water per gram of metal oxides and 0.6 mL water per gram of metal oxides, such as between 0.3 mL water per gram of metal oxides and 0.5 mL water per gram of metal oxides, or between 0.3 mL water per gram of metal oxides and 0.4 mL water per gram of metal oxides. In some embodiments, the slurry has a ratio of water to metal oxides between 0.35 mL water per gram of metal oxides and 0.45 mL water per gram of metal oxides.

In some embodiments, the slurry has a ratio of water to metal oxides of 0.1 mL water per gram of metal oxides, 0.2 mL water per gram of metal oxides, 0.3 mL water per gram of metal oxides, 0.4 mL water per gram of metal oxides, 0.5 mL water per gram of metal oxides, 0.6 mL water per gram of metal oxides, 0.7 mL water per gram of metal oxides, 0.8 mL water per gram of metal oxides, 0.9 mL water per gram of metal oxides, 1 mL water per gram of metal oxides, 2 mL water per gram of metal oxides, 3 mL water per gram of metal oxides, 4 mL water per gram of metal oxides, or 5 mL water per gram of metal oxides.

In some embodiments, the slurry has a ratio of water to metal oxides of 0.25 mL water per gram of metal oxides, 0.26 mL water per gram of metal oxides, 0.27 mL water per gram of metal oxides, 0.28 mL water per gram of metal oxides, 0.29 mL water per gram of metal oxides, 0.30 mL water per gram of metal oxides, 0.31 mL water per gram of metal oxides, 0.32 mL water per gram of metal oxides, 0.33 mL water per gram of metal oxides, 0.34 mL water per gram of metal oxides, 0.35 mL water per gram of metal oxides, 0.36 mL water per gram of metal oxides, 0.37 mL water per gram of metal oxides, 0.38 mL water per gram of metal oxides, 0.39 mL water per gram of metal oxides, 0.40 mL water per gram of metal oxides, 0.41 mL water per gram of metal oxides, 0.42 mL water per gram of metal oxides, 0.43 mL water per gram of metal oxides, 0.44 mL water per gram of metal oxides, 0.45 mL water per gram of metal oxides, 0.46 mL water per gram of metal oxides, 0.47 mL water per gram of metal oxides, 0.48 mL water per gram of metal oxides, 0.49 mL water per gram of metal oxides, 0.50 mL water per gram of metal oxides, 0.51 mL water per gram of metal oxides, 0.52 mL water per gram of metal oxides, 0.53 mL water per gram of metal oxides, 0.54 mL water per gram of metal oxides, or 0.55 mL water per gram of metal oxides.

The method disclosed herein can further include a process for controlled size modification of the metal oxides and the bismuth compound. For example, grinding, wet milling, dry milling, or crushing the metal oxides and the bismuth compound. The controlled size modification process may reduce the size of the metal oxides and the bismuth compound, which can improve their reactivity, or may allow for agglomeration of the metal oxides and/or the bismuth compound, which can allow for the creation of a powder or granule with low dusting properties and improve the powder's loading into equipment (for example, improved powder flowability or granulating).

In some embodiments, the metal oxides and the bismuth compound each have a particle size of less than 1 mm, such as less than 60 mesh (250 μm). For example, the metal oxides and the bismuth compound each may have a particle size in the range of from 0.5 μm to 250 μm, or from 1 μm to 200 μm, or from 1 μm to 150 μm, or from 1 μm to 100 μm, or from 1 μm to 50 μm, or from 10 μm to 200 μm, or from 10 μm to 150 μm, or from 10 μm to 100 μm, or from 10 μm to 50 μm, or from 50 μm to 200 μm, or from 50 μm to 150 μm, or from 50 μm to 100 μm.

In some embodiments, the method further includes a process for controlled size modification of the reducing agent. For example, grinding, wet milling, dry milling, or crushing the reducing agent.

Any suitable reducing agent may be included in the slurry. As used herein, the term “reducing agent” refers to a chemical substance that is capable of reducing an oxidation state of one or more of the metals of the metal oxides or the bismuth compound in the slurry. Suitable reducing agents to facilitate the reaction include reducing agents that are prone to decomposition or oxidation during the reaction process.

In some embodiments, the reducing agent includes an alcohol, a carboxylic acid, an ester, or a metal oxide. In some embodiments, the reducing agent includes an alcohol, a carboxylic acid, or an ester. Suitable examples of alcohol reducing agents include but are not limited to ethanol, methanol, reducing sugars, and polyols such as glycol and glycerol. Suitable examples of carboxylic acid reducing agents include but are not limited to oxalic acid, formic acid, acetic acid, and citric acid. Suitable examples of ester reducing agents include but are not limited to ethyl acetate, dimethyl carbonate, dimethyl oxalate, and diethyl oxalate. Suitable metal oxides include vanadium (IV) oxide. In some embodiments, the reducing agent is oxalic acid or ethanol. In some embodiments, the reducing agent is oxalic acid. In some embodiments, the reducing agent is ethanol.

The slurry can include one reducing agent, or two or more reducing agents. In some embodiments, the slurry includes one or more reducing agents. In some embodiments, the slurry includes no more than one reducing agent. The method disclosed herein can provide the advantage of a simplified procedure having a single reducing agent compared to previously reported methods that utilize more than one reducing agent.

The amount of reducing agent used in the slurry may be chosen based in part on the nature of the reducing agent being used. In some embodiments, a ratio of reducing agent used in the slurry to metal oxides used in the slurry is between 0.05 g of reducing agent per gram of metal oxides and 1 g of reducing agent per gram of metal oxides. The phrase “reducing agent” in the expression “a ratio of reducing agent used in the slurry to the amount of metal oxides used in the slurry” as used herein refers to the total mass of reducing agent used in the slurry, which may be the mass of one reducing agent, the mass of no more than one reducing agent, or the combined mass of two or more reducing agents. The phrase “metal oxides in the slurry” in the expression “a ratio of reducing agent used in the slurry to metal oxides used in the slurry” as used herein refers to the total mass of metal oxides used in the slurry, which includes an oxide of molybdenum; an oxide of vanadium; and one or both of an oxide of tantalum or an oxide of niobium. In embodiments where the reducing agent is a metal oxide, the total mass of metal oxides used in the slurry does not include the mass of the metal oxide being used as the reducing agent.

For example, a slurry comprising 1.5025 g of oxalic acid as the reducing agent, 5.359 g of MoO₃, 1.0674 g V₂O₅, and 0.4075 g of Ta₂O₅·xH₂O would have 6.8339 total metal oxides and a ratio of total reducing agent used in the slurry to total metal oxides used in the slurry of 0.22.

In some embodiments, the ratio of reducing agent used in the slurry to metal oxides in the slurry is between 0.1 g of reducing agent per gram of total metal oxides and 0.5 g of reducing agent per gram of metal oxides. In some embodiments, the ratio of reducing agent used in the slurry to metal oxides used in the slurry is between 0.15 g of reducing agent per gram of metal oxides and 0.25 g of reducing agent per gram of metal oxides. In a non-limiting example, the amount of oxalic acid used in the slurry is between 1 g and 2 g and the amount of metal oxides used in the slurry is between 6 g and 8 g. In another non-limiting example, the amount of oxalic acid used in the slurry is between 12 g and 14 g and the amount of metal oxides used in the slurry is between 68 g and 72 g.

In some embodiments of the methods disclosed herein, the metal oxides include an oxide of molybdenum, an oxide of vanadium, and an oxide of tantalum. In some embodiments, the slurry includes MoO₃, V₂O₅, Ta₂O₅·xH₂O, and Bi(OH)₃. In some embodiments, the slurry includes MoO₃, V₂O₅, Ta₂O₅·xH₂O, and Bi(OH)₃ in a mass ratio of MoO₃:V₂O₅:Ta₂O₅·xH₂O:Bi(OH)₃ of 1 g MoO₃: 0.1 to 0.3 g V₂O₅: 0.01 g to 0.10 g Ta₂O₅·xH₂O: 0.01 g to 0.20 g Bi(OH)₃. This mass ratio can be used at any suitable reaction scale. For example, the slurry may include from 4 to 6 g MoO₃, from 0.5 g to 1.5 g V₂O₅, from 0.2 g to 0.6 g Ta₂O₅·xH₂O, and from 0.05 to 0.2 g Bi(OH)₃. In another non-limiting example, the slurry may include from 20 to 25 g MoO₃, from 3 to 6 g V₂O₅, from 1 g to 3 g Ta₂O₅·xH₂O, and from 1 to 3 g Bi(OH)₃.

In some embodiments, the metal oxides include an oxide of molybdenum, an oxide of vanadium, and an oxide of niobium. In some embodiments, the slurry includes MoO₃, V₂O₅,Nb₂O₅ xH₂O, and Bi(OH)₃. In some embodiments, the slurry includes MoO₃, V₂O₅, Nb₂O₅·xH₂O, and Bi(OH)₃ in a mass ratio of MoO₃:V₂O₅:Nb₂O₅·xH₂O:Bi(OH)₃ of Ig MoO₃: 0.1 to 0.3 g V₂O₅: 0.01 g to 0.10 g Nb₂O₅·xH₂O: 0.01 g to 0.20 g Bi(OH)₃. This mass ratio can be used at any suitable reaction scale. For example, the slurry may include from 4 to 6 g MoO₃, from 0.5 g to 1.5 g V₂O₅, from 0.2 g to 0.6 g Nb₂O₅·xH₂O, and from 0.05 to 0.2 g Bi(OH)₃.

In some embodiments, the slurry is essentially free of a tellurium compound. The term “essentially free” as used herein means less than 10 ppm, less than 5 ppm, less than 2 ppm, or less than 1 ppm.

In some embodiments, the slurry is essentially free of a strong acid. The skilled person will appreciate that a strong acid is an acid that is completely or nearly completely ionized in a solution. For example, in some embodiments, the slurry is essentially free of nitric acid.

In the methods disclosed herein, the catalyst is formed in a hydrothermal synthesis reaction by heating the slurry. Any suitable reaction vessel may be used for the hydrothermal synthesis reaction. In some embodiments, the slurry is heated in a hydrothermal synthesis vessel. In some embodiments, the slurry is formed in a hydrothermal synthesis vessel and subsequently heated in the hydrothermal synthesis vessel. In some embodiments, the slurry is transferred to a hydrothermal synthesis vessel after the slurry is formed, and then heated in the hydrothermal synthesis vessel.

As used herein, the term “hydrothermal synthesis vessel” refers to a reaction vessel suitable for carrying out a reaction at elevated temperature and pressure, including but not limited to an autoclave, a digestion tank, a pressure vessel, a hydrothermal synthesis reactor, or a polytetrafluoroethylene (PTFE) high-pressure tank. In some embodiments, the hydrothermal synthesis vessel is an autoclave.

The slurry can be heated by ramping a temperature of the slurry and subsequently holding a temperature of the slurry. The ramping of the temperature can be used to avoid surface boiling of the slurry. The expression “ramping a temperature”, as used herein, refers to changing from an initial temperature to a final temperature over a time. For example, the temperature of the slurry may be ramped from an initial temperature of room temperature to a final temperature greater than room temperature over the course of a specified number of hours. The final temperature may be the holding temperature. The term “room temperature” as used herein refers to a temperature between 15° C. and 28° C. The term “holding temperature,” as used herein, refers to the temperature at which the reaction vessel is held, which can be measured by the ambient temperature of the oven the reaction vessel was placed in.

In some embodiments, the slurry is heated by ramping a temperature from ambient to a temperature between 100° C. and 200° C. over a ramping time between 2 hours and 48 hours; and holding the temperature at a holding temperature between 100° C. and 200° C. for a holding time between 12 hours and 160 hours. In some embodiments, the slurry is heated by ramping a temperature from ambient to a temperature between 100° C. and 200° C. over a ramping time between 2 hours and 48 hours; and holding the temperature at a holding temperature between 100° C. and 200° C. for a holding time between 12 hours and 120 hours. In some embodiments, the slurry is heated by ramping a temperature from ambient to a temperature between 150° C. and 200° C. over a ramping time between 2 hours and 24 hours; and holding the temperature at a holding temperature between 150° C. and 200° C. for a holding time between 24 hours and 60 hours.

In a non-limiting example, the slurry is heated by ramping a temperature from ambient to 180° C. over 12 hours; and holding the temperature at 180° C. for a holding time of 48 hours. In another non-limiting example, the slurry is heated by ramping a temperature from ambient to 180° C. over 24 hours; and holding the temperature at 180° C. for a holding time of 60 hours. In another non-limiting example, the slurry is heated by ramping a temperature from ambient to 180° C. over 2 hours; and holding the temperature at 180° C. for a holding time of 48 hours. In another non-limiting example, the slurry is heated by ramping a temperature from ambient to 180° C. over 24 hours; and holding the temperature at 180° C. for a holding time of 156 hours.

In some embodiments, the method further comprises washing the catalyst with water. For example, the catalyst may be washed with water until the filtrate is colorless. In some embodiments, the method further comprises a step of drying the catalyst, for example, at temperatures below 100° C. The drying can be done by any suitable method including, for example, at ambient temperature for a suitable time or in an oven overnight at, for example, a temperature of 90° C.

The method can further include calcining the catalyst to form a calcined catalyst. The skilled person will be familiar with suitable methods for calcining the catalyst. In some embodiments, the catalyst is calcined by placing the catalyst in a furnace under an oxygen-free environment; ramping a temperature of the furnace from ambient to a temperature between 500° C. and 620° C. over a ramping time between 2 hours and 10 hours; and holding the temperature of the furnace at a holding temperature between 500° C. and 620° C. for a holding time between 1 hour and 10 hours. For example, the catalyst is calcined by placing the catalyst in a furnace; ramping a temperature of the furnace from ambient to a temperature of 600° C. over a ramping time of 6 hours; and holding the temperature of the furnace at a holding temperature of 600° C. for a holding time of 2 hours.

As used herein, an “oxygen-free environment” refers to an environment having a molecular oxygen content below 10 ppm. For example, the furnace may be under an inert atmosphere, such as a purified nitrogen atmosphere or a purified argon atmosphere, or the furnace may be under a CO₂ and/or steam atmosphere.

In some embodiments, the ratio of the water in the slurry to amount of calcined catalyst is between 0.1 mL water per gram of calcined catalyst and 10 mL water per gram of calcined catalyst, such as between 0.1 mL water per gram of calcined catalyst and 5 mL water per gram of calcined catalyst, between 0.1 mL water per gram of calcined catalyst and 4 mL water per gram of calcined catalyst, between 0.1 mL water per gram of calcined catalyst and 3 mL water per gram of calcined catalyst, between 0.1 mL water per gram of calcined catalyst and 2 mL water per gram of calcined catalyst, or between 0.1 mL water per gram of calcined catalyst and 1 mL water per gram of calcined catalyst.

The phrase “amount of calcined catalyst” as used herein refers to the mass of the catalyst after all the catalyst obtained from catalyst synthesis has undergone calcination, such as the calcination described herein. In a non-limiting example, the ratio of the water in the slurry to amount of calcined catalyst formed may be between 0.3 mL water per gram of calcined catalyst and 0.8 mL water per gram of calcined catalyst, between 0.4 mL water per gram of calcined catalyst and 0.6 mL water per gram of calcined catalyst, or 0.5 mL water per gram of calcined catalyst.

In some embodiments, the ratio of water in the slurry to amount of calcined catalyst is 0.1 mL water per gram of calcined catalyst, 0.2 mL water per gram of calcined catalyst, 0.3 mL water per gram of calcined catalyst, 0.4 mL water per gram of calcined catalyst, 0.5 mL water per gram of calcined catalyst, 0.6 mL water per gram of calcined catalyst, 0.7 mL water per gram of calcined catalyst, 0.8 mL water per gram of calcined catalyst, 0.9 mL water per gram of calcined catalyst, 1 mL water per gram of calcined catalyst, 2 mL water per gram of calcined catalyst, 3 mL water per gram of calcined catalyst, 4 mL water per gram of calcined catalyst, or 5 mL water per gram of calcined catalyst.

In some embodiments, the ratio of water in the slurry to amount of calcined catalyst formed is 0.40 mL water per gram of calcined catalyst, 0.41 mL water per gram of calcined catalyst, 0.42 mL water per gram of calcined catalyst, 0.43 mL water per gram of calcined catalyst, 0.44 mL water per gram of calcined catalyst, or 0.45 mL water per gram of calcined catalyst, 0.46 mL water per gram of calcined catalyst, 0.47 mL water per gram of calcined catalyst, 0.48 mL water per gram of calcined catalyst, 0.49 mL water per gram of calcined catalyst, 0.50 mL water per gram of calcined catalyst, 0.51 mL water per gram of calcined catalyst, 0.52 mL water per gram of calcined catalyst, 0.53 mL water per gram of calcined catalyst, 0.54 mL water per gram of calcined catalyst, 0.55 mL water per gram of calcined catalyst, 0.56 mL water per gram of calcined catalyst, or 0.57 mL water per gram of calcined catalyst, 0.58 mL water per gram of calcined catalyst, 0.59 mL water per gram of calcined catalyst, 0.60 mL water per gram of calcined catalyst, 0.61 mL water per gram of calcined catalyst, 0.62 mL water per gram of calcined catalyst, or 0.63 mL water per gram of calcined catalyst, 0.64 mL water per gram of calcined catalyst, or 0.65 mL water per gram of calcined catalyst.

In some embodiments, a ratio of a total amount of water used to prepare the catalyst is between 0.1 mL water per gram of catalyst and 60 mL water per gram of catalyst. In some embodiments, a ratio of a total amount of water used to prepare the catalyst is between 0.1 mL water per gram of catalyst and 25 mL water per gram of catalyst. As used herein, a “total amount of water used to prepare the catalyst” includes the water in the slurry, as well as any water used in any other steps, such as the optional step of washing the catalyst with water, and/or any water used to transfer materials between reaction vessels. In some embodiments, a ratio of a total amount of water used to prepare the catalyst is between 0.1 mL water per gram of catalyst and 20 mL water per gram of catalyst, 0.5 mL water per gram of catalyst and 15 mL water per gram of catalyst, 1 mL water per gram of catalyst and 10 mL water per gram of catalyst, 5 mL water per gram of catalyst and 10 mL water per gram of catalyst, between 0.1 mL water per gram of catalyst and 5 mL water per gram of catalyst, or between 0.1 mL water per gram of catalyst and 1 mL water per gram of catalyst. In some embodiments, a ratio of a total amount of water used to prepare the catalyst is between 0.2 mL water per gram of catalyst and 10 mL water per gram of catalyst.

The catalysts prepared from the methods disclosed herein include molybdenum (Mo); vanadium (V); bismuth (Bi); tantalum (Ta) or niobium (Nb), or both; and oxygen (O). In some embodiments, the catalyst has a formula selected from MoₐV_{b}Bi_{c}Ta_{d}Oₓ, and MoₐV_{b}Bi_{c}Nb_{d}Oₓ.

In some embodiments, the methods for preparing a catalyst disclosed herein may further include combining the calcined catalyst with one or more of a solid support, carrier, binder, and lubricant, such as the solid supports, carriers, binders, and lubricants disclosed elsewhere herein, to provide a catalyst material. In some embodiments, the catalyst material is prepared by a method that includes preparing an aqueous mixture that includes (i) a catalyst disclosed herein; (ii) a solid support or carrier, and (iii) a lubricant and/or a binder. In some embodiments, the method can further include removing a substantial amount of the water (for example, from 50 wt. % to 99 wt. %) from the aqueous mixture, such as, for example, by heating the mixture at a temperature from 50° C. to 100° C. In some embodiments, the method includes forming the catalyst material into a formed catalyst materials such as, for example, a pelleted catalyst material.

The catalysts disclosed herein may be suitable as catalysts in oxidative dehydrogenation reactions. The present disclosure further provides a process for the oxidative dehydrogenation of ethane to ethylene in an oxidative dehydrogenation reactor with any of the oxidative dehydrogenation catalyst described herein. The catalyst may be included in any of the catalyst materials described herein.

As used herein, the term “oxidative dehydrogenation” or “ODH” refers to a process that couples the endothermic dehydrogenation of an alkane (CₙH₂ₙ₊₂) with the strongly exothermic oxidation of hydrogen as is further described herein to form, amongst other things, alpha-olefins. In some embodiments, the alkane is one or more of ethane, propane, butane, pentane, hexane, octane, decane, and dodecane. In some embodiments, the alkane is ethane or propane. In some embodiments, the alkane is ethane. For testing catalysts, the ODH reactions herein are assumed to be referring to the ODH of ethane.

In some embodiments, the catalyst has a 45% ethane conversion temperature from 300° C. to 420° C. In some embodiments, the catalyst has a 45% ethane conversion temperature from 300° C. to 400° C. In some embodiments, the catalyst has a 45% ethane conversion temperature from 325° C. to 390° C. In some embodiments, the catalyst has a 45% ethane conversion temperature from 340° C. to 390° C. In some embodiments, the catalyst has a 45% ethane conversion temperature from 350° C. to 400° C. In some embodiments, the catalyst has a 45% ethane conversion temperature from 350° C. to 380° C. In some embodiments, the catalyst has a 45% ethane conversion temperature from 360° C. to 375° C. As used herein, the phrase “45% ethane conversion temperature” refers to the temperature at which 45% of ethane in a gas stream is converted to a product other than ethane at a given fixed feed composition, weight hourly space velocity, and reactor inlet pressure. The 45% ethane conversion temperature of an oxidative dehydrogenation catalyst can be determined using a microreactor unit (MRU).

In a microreactor unit, the 45% ethane conversion temperature of a catalyst can be determined by passing a feed gas over a catalyst bed in a reactor tube. The MRU reactor tube has an outer diameter of 0.5 inches and an internal diameter of 0.4 inches and length of 15 inches. For example, the reactor tube can be stainless-steel SWAGELOK® Tubing with a wall thickness of 0.049 inches. The feed gas can include ethane and oxygen having a molar ratio of 70:30 to 90:10. For example, the feed gas can include ethane and oxygen having a molar ratio of 82:18. Alternatively, the feed gas can include ethane, oxygen, and nitrogen. The molar ratio of ethane to oxygen to nitrogen can be 18:8:74 to 54:18:28. For example, the molar ratio of ethane to oxygen to nitrogen can be 20:10:70. The flow rate of the feed gas can be 70 standard cubic centimeters per minute (sccm) to 80 sccm. For example, the flow rate of the feed gas can be 75 sccm (e.g., 74.6 sccm). The catalyst bed consists of the oxidative dehydrogenation catalyst and a filler, such as quartz sand, 1:0.5 to 1:3 volume ratio, with the total weight for the oxidative dehydrogenation catalyst being 1.96 to 2.00 g. Any remaining space in the reactor tube (e.g., below or above the catalyst bed) is packed with an additional filler, such as quartz sand. The 45% ethane conversion temperature is determined at a weight hourly space velocity (WHSV) of 3.57 h⁻¹, with the WHSV based on the weight of catalyst in the sample, and a gas hourly space velocity (GHSV) of 2,000 to 5,000 h⁻¹. As used herein, the expression “weight hourly space velocity” refers to the weight flow of the total feed gas divided by the weight of the catalyst. Typically, the inlet pressure is in the range of 1 pound per square inch gauge (psig) to 2.5 psig and the outlet pressure is in the range of 0 psig to 0.5 psig. The gas feed exiting the catalyst bed is analyzed by gas chromatography to determine the percent of various hydrocarbons (e.g., ethane and ethylene) and, optionally other gases such as O₂, CO₂, and CO.

Conversion of the ethane feed gas to products by the ODH process is calculated as a volume flow rate change of ethane in the product compared to feed ethane volume flow rate using the following formula:

$$C = ( \frac{2 * {X}_{Ethylene} + {X}_{CO 2} + {X}_{CO} + 2 * {X}_{Acetic Acid}}{2 * {X}_{Ethylene} + 2 * {X}_{Ethane} + {X}_{CO 2} + {X}_{CO} + 2 * {X}_{Acetic Acid}} ) * 100 \% \qquad Eq . 1$$

In Equation 1, C is the percent (molar percent) of ethane feed gas that has been converted from ethane to another product (that is, ethane conversion) and X is the molar concentration of the corresponding compound in the gaseous effluent exiting the reactor at corresponding temperature.

Furthermore, the gas exiting the reactor can be analyzed by gas chromatography to determine catalyst or catalyst material selectivity to ethylene (that is, the percentage on a molar basis of ethane that forms ethylene). Selectivity to ethylene can be determined using the following equation:

$${S}_{Ethylene} = ( \frac{2 * {X}_{Ethylene}}{2 * {X}_{Ethylene} + {X}_{CO 2} + {X}_{CO} + 2 * {X}_{Acetic Acid}} ) * 100 \% \qquad Eq . 2$$

In Equation 2, S_{Ethylene} is the selectivity to ethylene and X is the molar concentration of the corresponding compound in the gaseous effluent exiting the reactor at corresponding temperature. As used herein, the phrase “selectivity to ethylene” refers to the percentage on a molar basis of converted or reacted ethane that forms ethylene.

In some embodiments, the catalyst has a selectivity to ethylene from 65% to 99%. In some embodiments, the catalyst material has a selectivity to ethylene from 75% to 95%. In some embodiments, the catalyst has a selectivity to ethylene from 83% to 93%. In some embodiments, the catalyst has an ethylene selectivity of at least 90% at an ethane conversion of at least 45%.

In some embodiments, the catalyst has a selectivity to acetic acid of less than 38 mol. % in a process for the oxidative dehydrogenation of ethane to ethylene. In some embodiments, the catalyst has a selectivity to acetic acid of less than 25 mol. %. For example, the catalyst material can have a selectivity to acetic acid of 1 mol. % to 15 mol. %, 3 mol. % to 12 mol. %, or 7 mol. % to 12 mol. % in a process for the oxidative dehydrogenation of ethane to ethylene. In some embodiments, the catalyst material has a selectivity to acetic acid of 1 mol. %, 2 mol. %, 3 mol. %, 4 mol. %, 5 mol. %, 6 mol. %, 7 mol. %, 8 mol. %, 9 mol. %, 10 mol. %, 11 mol. %, 12 mol. % or 13 mol. % in a process for the oxidative dehydrogenation of ethane to ethylene. As used herein, the phrase “selectivity to acetic acid” refers to the percentage on a molar basis of converted or reacted ethane that forms acetic acid.

GHSV (gas hourly space velocity) is defined as volumetric flow of the reactor feed gas divided by the volume of the catalyst bed. As used herein, the term “volume of the catalyst bed” refers to the volume occupied by catalyst particles, optional diluent particles, and any void spaces within the catalyst bed. For GHSV values of Catalyst Materials, the catalyst bed is treated as catalyst only (not including support) where an assumption is made that the total volume of the catalyst material measured when multiplied by the wt. % of catalyst is the volume of the catalyst. The GHSV can be calculated based off the measured volume of the pressed particles (before mixing with quartz sand) and varies depending on each catalyst or catalyst material bulk density. For catalyst materials discussed herein, the GHSV reported is for the catalyst only, where an assumption was made that the total volume of the catalyst material measured when multiplied by the wt. % of catalyst is the volume of the catalyst.

Ethylene provided by ODH of ethane using the catalyst, catalyst materials, and processes described herein can subsequently be converted into a variety of products. For example, ethylene can be converted to very low density polyethylene (VLDPE), low density polyethylene (LDPE), linear low density polyethylene (LLDPE), medium density polyethylene (MDPE), high density polyethylene (HDPE), ethylene dichloride, ethylene oxide, ethylbenzene, linear alcohols, vinyl acetate, alkanes, alpha olefins (e.g., 1-hexene and 1-octene), various hydrocarbon-based fuels, ethanol and the like. These products can then be further processed using methods well known to one of ordinary skill in the art to obtain other valuable chemicals and consumer products.

In some embodiments, ethylene provided by the ODH process described herein is converted to polyethylene. In some embodiments, the polyethylene is very low density polyethylene (VLDPE), low density polyethylene (LDPE), linear low density polyethylene (LLDPE), medium density polyethylene (MDPE), and high density polyethylene (HDPE).

Notwithstanding that the numerical ranges and parameters setting forth the broad scope of the disclosure are approximations, the numerical values set forth in the specific examples are reported as precisely as possible. Any numerical values, however, inherently contain certain errors necessarily resulting from the standard deviation found in their respective testing measurements.

In addition, it should be understood that any numerical range recited herein is intended to include all sub-ranges subsumed therein. For example, a range of “1 to 10” or “between 1 and 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values. Unless expressly indicated otherwise, the various numerical ranges specified in this application are approximations.

In this document, the terms “a”, “an”, or “the” are used to include one or more than one unless the context clearly dictates otherwise. The term “or” is used to refer to a nonexclusive “or” unless otherwise indicated. The statement “at least one of A and B” has the same meaning as “A, B, or A and B”. In addition, it is to be understood that the phraseology or terminology employed herein, and not otherwise defined, is for the purpose of description only and not of limitation.

### EXAMPLES

### Reagents

Molybdenum(VI) oxide (MoO₃), vanadium(V) oxide (V₂O₅), tellurium dioxide (TeO₂) and oxalic acid dihydrate were purchased from Sigma-Aldrich. Bismuth(III) hydroxide (Bi(OH)₃) was purchased from Alfa Aesar/Fisher Scientific Canada. The tantalum pentoxide hydrate (Ta₂O₅·xH₂O) was purchased from BassTech International. The x in Ta₂O₅·xH₂O was 2.57 as measured by thermogravimetric analysis. The niobium pentoxide hydrate (Nb₂O₅·xH₂O) was purchased from Companhia Brasileira de Metalugia e Mineração. The x in Nb₂O₅·xH₂O was 4.57 as measured by thermogravimetric analysis. Alpha alumina was purchased from Fisher Scientific Canada. All reagents were used as is without any further purification. All water used was distilled deionized water.

### Preparation of Comparative Sample 1C

Comparative Sample 1C was prepared with the solid reagents listed in Table 1.

TABLE 1
Amounts of Reagents Used for Sample 1C.

| Reagent | Amount | |
|---|---|---|
| MoO₃ | 5.3707 | g |
| V₂O₅ | 1.0310 | g |
| TeO₂ | 0.3033 | g |
| Ta₂O₅•xH₂O | 0.4192 | g |
| Oxalic acid dihydrate | 1.3167 | g |
| Distilled water | 3 | mL |

The solid reagents were mixed and lightly ground using a mortar and pestle. The solid mixture was then transferred into an 8 mL glass vial, after which 2 mL of the distilled water solvent was added. The sample was stirred lightly with a glass stir rod to form a thick orange slurry and another 1 mL of water was then used to rinse sample stuck to the stir rod back into the vial. The vial was then placed in a glass lined steel autoclave and water was filled around the vial to the level of the solids to help with heat transfer and to maintain a humid atmosphere in the vessel. The autoclave was then sealed and placed in an oven to heat from room temperature to 180° C. over 12 hours, held at 180° C. for 48 hours, then turned off to cool back to ambient temperature over 3-4 hours.

After the reaction, the sample was a deep-purple, hard solid that had increased in volume by approximately 1.5×. The sample was scraped from the vial onto filter paper in a vacuum filtration set-up and were washed with approximately 50 mL of distilled water, with the filtrate being a clear, deep blue color. The sample was washed until the filtrate from the sample was colorless, then was left to dry on the filter paper to obtain shiny purple-black powdered solid. The solid was then calcined in a tubular autoclave under N₂ flow (linear velocity at standard temperature and pressure (STP) of 3.9 cm/min, 0.25 ppm (vol.) of residual oxygen) for 12 hours at 60° C., after which it was heated to 600° C. over 6 hours, held at 600° C. for 2 hours, then the furnace was turned off and the sample cooled back to ambient temperature over approximately 12 hours. After calcination, the sample was a deep purple-black powder. The mass of the solid sample before calcination was 8.0214 g and after calcination was 5.9457 g.

### Preparation of Examples 2E-5E

### Preparation of Catalysts

Four separate examples, 2E-5E, were prepared with solid reagents listed in Table 2. For each example, the solid reagents were mixed and lightly ground using a mortar and pestle. The solid mixtures were then transferred into individual glass vials, after which a portion of the distilled water solvent was added. The sample was stirred lightly with a glass stir rod to form a thick orange slurry and another portion (~1 mL) of water was then used to rinse sample stuck to the stir rod back into the vial (total water used to form the slurry and rinse the sample is shown in Table 2).

TABLE 2
Amounts of Reagents Used to Prepare Slurry for Examples
2E-5E, and Mass of Each Catalyst After Calcination.

| | 2E | 3E | 4E | 5E |
|---|---|---|---|---|
| MoO₃ (g) | 5.3817 | 5.3590 | 5.3673 | 21.4624 |
| V₂O₅ (g) | 1.0505 | 1.0674 | 1.0419 | 4.1691 |
| Ta₂O₅•xH₂O (g) | — | 0.4075 | 0.4255 | 1.7249 |
| Nb₂O₅•xH₂O (g) | 0.3206 | — | — | — |
| Bi(OH)₃ (g) | 0.5133 | 0.5125 | 0.5011 | 2.0193 |
| Oxalic acid dihydrate (g) | 1.5083 | 1.5025 | 1.3066 | 5.2440 |
| Distilled water (mL) | 3.5 | 3.5 | 2.7 | 10.8 |
| Mass before calcination (g) | 10.5580^{a} | 8.9112 | n.d.^{b} | 24.6823 |
| Mass after calcination (g) | 6.5890 | 6.3501 | n.d.^{b} | 23.3152 |

^{a}Sample was still wet
^{b}Not determined.

Respective vials for each example 2E-5E were placed together in a glass lined steel autoclave, along with a blank reference vial filled with water, and water was filled around the vials to the level of the solids to help with heat transfer and to maintain a humid atmosphere in the vessel. The autoclave was a 300 mL PARR reactor, available from Parr Instrument Company of Moline, IL, USA (Head of assembly serial number: 453HC T316 091902 24820B; Body of assembly serial number: 452HC T316 091902 24820A).

The autoclave was sealed and placed in an oven to heat from room temperature to 180° C. over 12 hours, held at 180° C. for 48 hours, then turned off to cool back to ambient over 3 to 4 hours. After the reaction, the samples were deep purple hard solid samples that had increased in volume by approximately 1.5×. The samples were scraped from the vials onto respective filter papers, each in a vacuum filtration set-up, and were each washed with approximately 50 mL of distilled water, with the filtrate being a clear faintly blue color for sample 2E and a deep blue for samples 3E and 4E. Each sample was washed until the filtrate was colorless, then was left to dry on the filter paper to obtain shiny purple-black powdered solid.

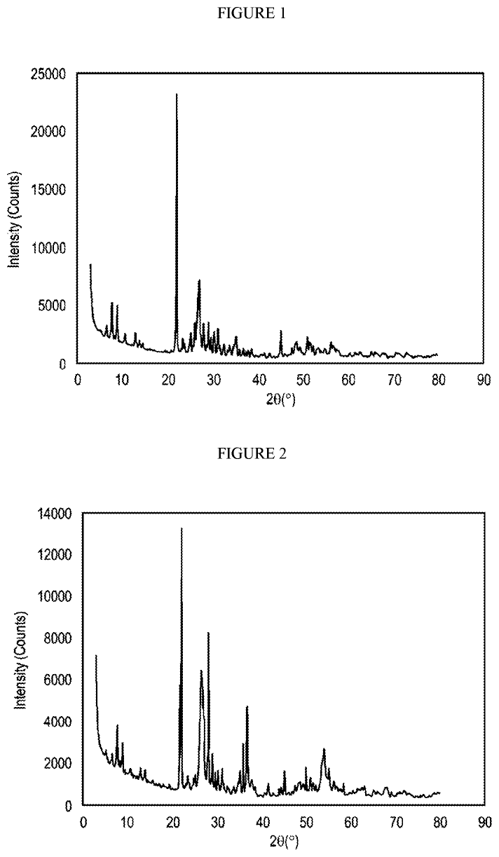

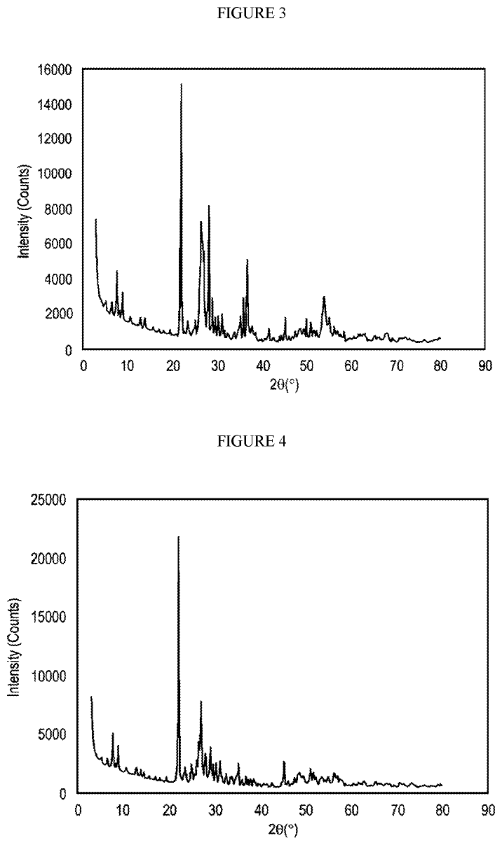

The solids for each example 2E to 5E were then calcined in a tubular autoclave under N₂ flow for 12 hours at 60° C., after which they were heated to 600° C. over 6 hours, held at 600° C. for 2 hours, then the furnace was turned off and the samples cooled back to ambient conditions over approximately 12 hours. The mass of each example catalyst 2E to 5E after calcination is shown in Table 2. The PXRD patterns of examples 2E to 5E are shown in FIGS. 2 to 5.

### Preparation of Example 5E—Pelleted

For this procedure, a target of 50 wt. % catalyst phase and 50 wt. % alumina after sintering (as described herein) was used. An additional 2 wt. % poly(ethylene glycol) 1000 and 3 wt. % poly(vinyl alcohol) (MOWIOL® 8-88), along with 1000 ppm of poly(acrylic acid), were added to act as binding agents. All components shown in Table 3 were mixed in a beaker at 90° C. using an overhead stirrer until most of the water had evaporated leaving a thick purple paste.

TABLE 3
Amounts of Materials Used for Formulating Example 5E-Pelleted.

| | |
|---|---|
| 5-E Calcined (g) | 10.0671 |
| α-Al₂O₃ (g) | 10.0771 |
| PEG 1000 (g) | 0.3912 |
| Mowilol 8-88 (g) | 0.6008 |
| Poly(acrylic acid) (g) | 0.0163 |
| Water (mL) | 90 |

The beaker was then transferred into an oven to dry overnight at 90° C. After drying, the hard solid chunks were pulverized using a mortar and pestle, and the particles were sieved to obtain sizes between 180-500 μm. To the sieved particles, 1 wt. % of natural graphite flakes (>325 mesh) were added, and the mixture was shaken in a closed container to coat the granules. The graphite coated particles were then fed into a Dott Bonapace CPR-6 pellet press fitted with a 3 mm cylindrical die, and the die settings were adjusted to press cylindrical pellets with an axial crush strength >50 N. Pellet dimensions were approximately 3 mm diameter and 5 mm length.

The pellets were sintered in a two-step procedure. Firstly, under air flow (1.9 cm/min linear velocity at STP), the pellets were heated to 400° C. at 1.0° C./min, held at 400° C. for 1 hours, then cooled to room temperature over approximately 8 hours. The furnace was then purged with nitrogen for 8 hours flow (3.9 cm/min linear velocity at STP), after which it was heated to 600° C. at a rate of 1.6° C./min, then heating was stopped and the furnace was cooled to room temperature over approximately 12 hours. Axial crush strength of the catalyst pellets generally remained constant before and after the sintering step.

### Preparation of Examples 6E and 7E

Examples 6E and 7E were prepared with increased levels of Bi(OH)₃ and with washing (6E) or without washing (7E), and were synthesized following a similar procedure listed for samples 2E-5E. Amounts of reagents used are listed in Table 4. All solid components were added to a blender and mixed for 1 minute pulses four times to pulverize and blend the solids, shaking and tapping the sides of the container between pulses. The solid mixture was then transferred to a 60 mL disposable glass hypovial. Water was added and the mixture was stirred to form an orange slurry. The vial was then placed into a 300 mL steel autoclave with water filled around the outside of the vial to improve heat transfer and maintain 100% relative humidity inside the vessel. The autoclave was sealed and placed in an oven to heat from room temperature to 180° C. over 12 hours, held at 180° C. for 48 hours, then heating was stopped and the vessel was cooled back to room temperature passively over approximately 6 hours.

TABLE 4
Amounts of Reagents Used to Prepare
Slurry for Examples 6E and 7E.

| | |
|---|---|
| MoO₃ (g) | 53.6360 |
| V₂O₅ (g) | 10.4475 |
| Ta₂O₅•xH₂O (x = 2.57) (g) | 5.0007 |
| Bi(OH)₃ (g) | 6.2476 |
| Oxalic acid dehydrate (g) | 12.9960 |
| Distilled water (mL) | 20 |

After cooling, the autoclave was vented and the vial was removed. The orange slurry became a deep purple solid over the course of the reaction. The solid was pulverized and separated into two approximately equal portions. One of the portions was used as is, without any additional washing steps (example 6E), while the other was washed with water using a vacuum filtration set-up until the filtrate was clear and colorless (example 7E). Both samples were dried in an oven at 90° C. overnight to provide 31.8692 g of Example 6E and 38.1755 g of example 7E.

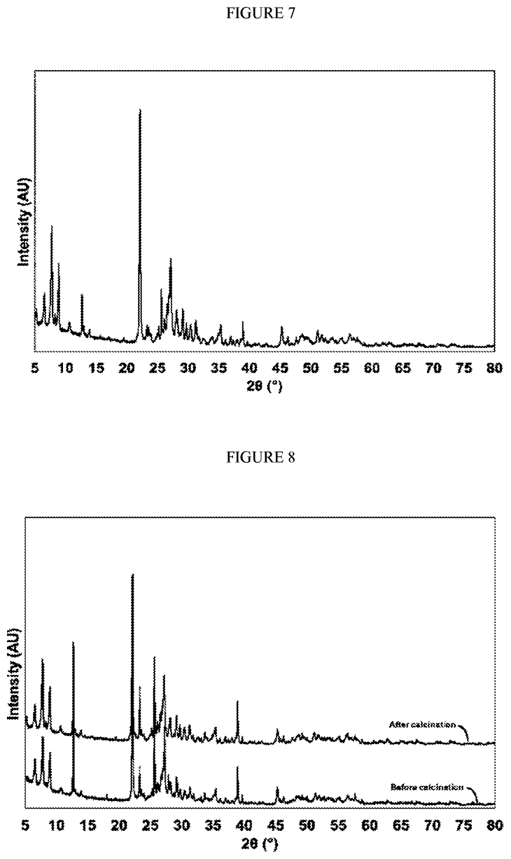

The solid samples were calcined under N₂ flow (3.9 cm/min linear velocity at STP) in a tubular quartz furnace. The solids were heated to 600° C. at a rate of 1.6° C./min, held at 600° C. for 2 hours, then heating was stopped and the solids were cooled passively to room temperature under N₂ flow for approximately 12 hours. After calcination 3.25% and 2.48% mass loss were observed for samples 6E and 7E, respectively. The PXRD patterns for example 6E and 7E after calcination are shown in FIGS. 6 and 7, respectively.

### Preparation of Example 8E

Example 8E was synthesized following the same procedure as example 6E using reagent amounts listed in the Table 5. After drying, 65.8639 g was obtained.

TABLE 5
Amounts of Reagents used to Prepare Slurry for Example 8E.

| | |
|---|---|
| MoO₃ (g) | 53.6751 |
| V₂O₅ (g) | 10.4217 |
| Ta₂O₅•xH₂O (x = 2.57) (g) | 4.0832 |
| Bi(OH)₃ (g) | 4.5792 |
| Oxalic acid dehydrate (g) | 12.9197 |
| Distilled water (mL) | 22 |

The solid sample was calcined under N₂ flow (3.9 cm/min linear velocity at STP) in a tubular quartz furnace. The solid was heated to 600° C. at a rate of 1.6° C./min, held at 600° C. for 2 hours, then heating was stopped and the solids were cooled passively to room temperature under N₂ flow for approximately 12 hours. After calcination 2.7% mass loss was observed. The PXRD pattern of example 8E before and after calcination is shown in FIG. 8.

### Preparation of Example 9E

Example 9E was prepared using a larger scale reaction that omitted the solid grinding step. All solids and water listed in Table 6 were added to a 1.8 L PTFE beaker and stirred with an overhead stirrer for 45 minutes to form an orange slurry. The beaker was then placed in a 2 L steel autoclave and 50 mL of water was filled around the outside of the PTFE beaker in order to maintain 100% relative humidity inside the vessel. The vessel was then sealed and placed in an oven to heat from room temperature to 180° C. over 12 hours, held at 180° C. for 48 hours, then heating was stopped and the vessel was cooled back to room temperature over approximately 6 hours. The vessel was then vented in a fume hood, and the dark purple solid was transferred into a 3 L beaker. Water (1 L) was added to the beaker, and the mixture was stirred with an overhear stirrer overnight. The purple slurry was then filtered by vacuum filtration and washed with a further 3 L of water in 1 L portions, then dried in an oven at 90° C. for 24 hours and 813.27 g of dry catalyst was obtained.

TABLE 6
Amounts of Reagents Used to Prepare Slurry for Example 9E.

| | |
|---|---|
| MoO₃ (g) | 600.05 |
| V₂O₅ (g) | 116.54 |
| Ta₂O₅•xH₂O (x = 2.57) (g) | 48.31 |
| Bi(OH)₃ (g) | 56.35 |
| Oxalic acid dehydrate (g) | 146.57 |
| Distilled water (mL) | 315 |
| Mass after synthesis (g) | 813.27 |

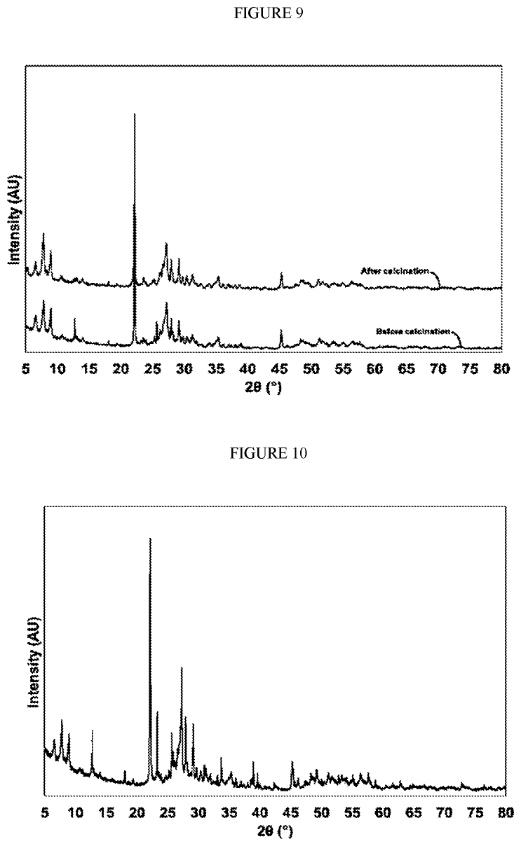

The dry catalyst 9E was calcined in a tubular quartz furnace under nitrogen flow (3.9 cm/min linear velocity at STP). After sufficient time was given for the furnace to purge with nitrogen (~8 hours), the furnace was heated from room temperature to 600° C. at 1.6° C./min, held at 600° C. for 2 hours, then the heating was stopped, and the sample was cooled back to room temperature over approximately 12 hours. The PXRD patterns for catalyst example 9E before and after calcination are shown in FIG. 9.

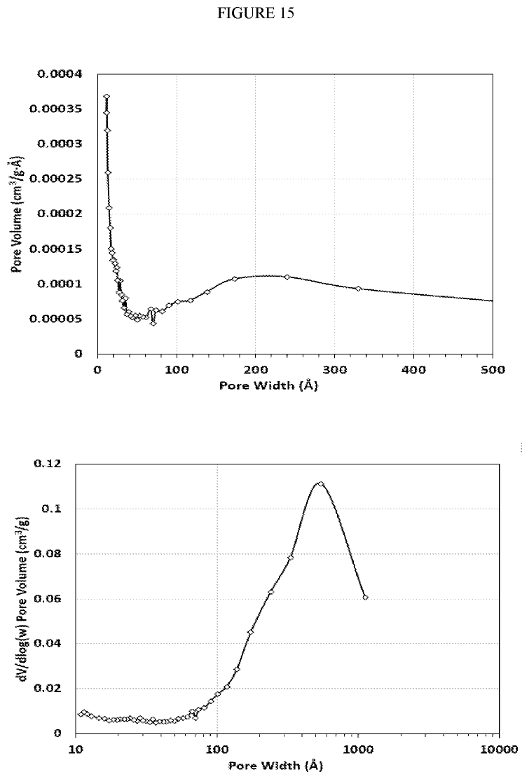

Nitrogen physisorption analysis was collected using a Micromeritics Instruments TriStar gas sorption analyzer. The powdered sample was loaded into a physisorption cell and degassed at 120° C. overnight (>12 h) prior to adsorption measurement. Nitrogen gas sorption analysis was collected at −196° C. using a liquid nitrogen bath. A Brunauer-Emmett-Teller (BET) model was used to determine the surface area of the sample. FIG. 15 shows a BJH plot of pore volume for example 9E. Total pore volume was calculated at the relative pressure P/P₀=0.99. A BET surface area of 17 m²/g and a pore volume of 0.14 cm³/g was measured.

### Preparation of Example 10E

Example 10E was synthesized generally following the same procedure as example 9E, except using an extended holding time at 180° C. of 156 hours (from 48 hours). The amounts of reagents used are listed in the Table 7.

TABLE 7
Amounts of Reagents Used to Prepare Slurry for Example 10E.

| | |
|---|---|
| MoO₃ (g) | 600.03 |
| V₂O₅ (g) | 116.58 |
| Ta₂O₅•xH₂O (x = 2.57) (g) | 48.45 |
| Bi(OH)₃ (g) | 56.45 |
| Oxalic acid dehydrate (g) | 146.57 |
| Distilled water (mL) | 350 |
| Mass after synthesis (g) | 813.48 |

### Preparation of Example 11E

The metal oxides and hydroxides from Table 8 were added to a blender and blended three time in 30 second pulses. The blended solids, oxalic acid and water were then added to a 1.8 L PTFE beaker and stirred with an overhead stirrer for 45 minutes to form an orange slurry. The beaker was then placed in a 2 L steel autoclave and 50 mL of water was filled around the outside of the PTFE beaker in order to maintain 100% relative humidity inside the vessel. The vessel was then sealed and placed in an oven to heat from room temperature to 180° C. over 12 hours, held at 180° C. for 48 hours, then heating was stopped and the vessel was cooled to room temperature over approximately 6 hours. The vessel was then vented in a fume hood, then the dark purple solid was transferred into a 3 L beaker. Water (1 L) was added to the beaker, and the mixture was stirred with an overhear stirrer overnight. The purple slurry was then filtered by vacuum filtration and washed with a further 3 L of water in 1 L portions, then was transferred into an oven at 90° C. to dry for 24 hours. The PXRD of example 11E before calcination is shown in FIG. 10.

TABLE 8
Amounts of Reagents Used to Prepare Slurry for Example 11E.

| | |
|---|---|
| MoO₃ (g) | 600.03 |
| V₂O₅ (g) | 116.57 |
| Ta₂O₅•xH₂O (x = 2.57) (g) | 48.21 |
| Bi(OH)₃ (g) | 56.46 |
| Oxalic acid dehydrate (g) | 146.64 |
| Distilled water (mL) | 330 |
| Mass after synthesis (g) | 799.03 |

The dry catalyst was then calcined in a tubular quartz furnace under nitrogen flow (3.9 cm/min linear velocity at STP). After the furnace was purged with nitrogen (~8 hours), the furnace was heated from room temperature to 600° C. at 1.6° C./min, held at 600° C. for 2 hours, then the heating was stopped, and the sample was cooled to room temperature over approximately 12 hours. A PXRD patter of example 11E is shown in FIG. 10.

### Instruments and Measurements

### Elemental Analysis

Catalyst metal compositions were determined by energy dispersive X-ray spectroscopy (EDX) for calcined samples 4E, 5E, 9E, and 11E. EDX was conducted using a JEOL JED-2300 DRY SDD™ EDX detector. The EDX scan was conducted on the largest rectangular area that was covered by the sample (approximately 2.8 mm×2.1 mm, typically at ~50× magnification but this can vary depending on sample size and coverage). The data analysis software was AnalysisStation provided by JEOL. The scan was conducted at 25 kV accelerating voltage. The results of EDX analysis for catalyst examples 4E, 5E, 9E, and 11E are shown in Table 9.

TABLE 9
EDX Analysis Results for Samples 4E, 5E, 9E, and 11E.

| Example | Metal Molar Ratios |
|---|---|
| 4E | Mo₁V_{0.32}Bi_{0.04}Ta_{0.03} |
| 5E | Mo₁V_{0.33}Bi_{0.05}Ta_{0.04} |
| 9E | Mo₁V_{0.26}Bi_{0.06}Ta_{0.03} |
| 11E | Mo₁V_{0.26}Bi_{0.05}Ta_{0.05} |

### Scanning Electron Microscopy (SEM)

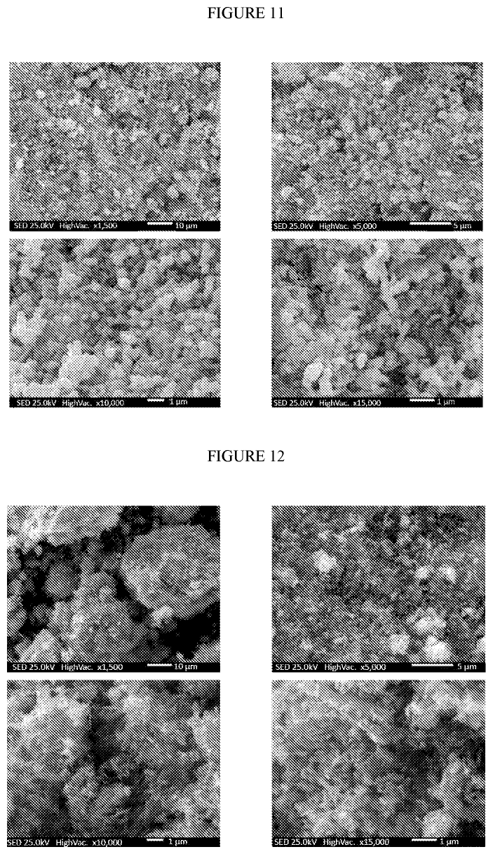

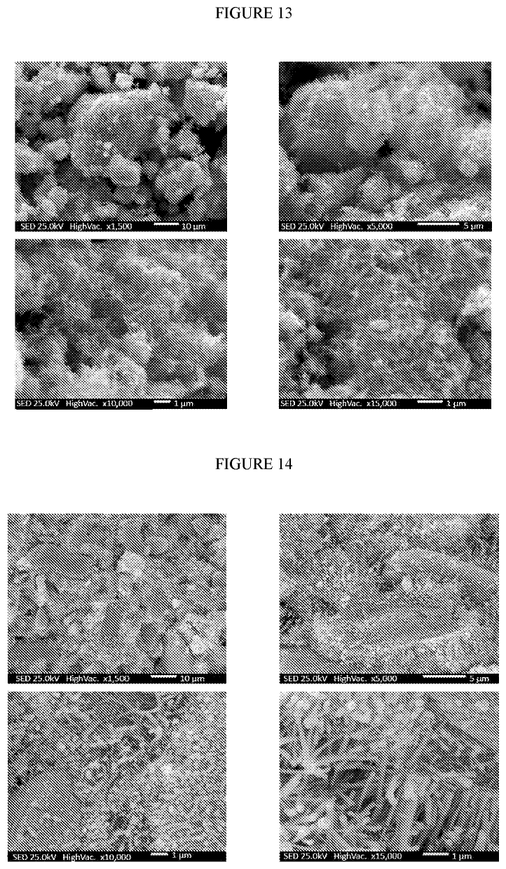

Scanning electron microscope (SEM) images were collected using a JEOL-JSM300 LV SEM. Samples were prepared on an aluminum stud with double sided carbon tape. FIGS. 11 through 14 show SEM images of examples 2E, 4E, 5E. and 11E, respectively.

### Powder X-ray Diffraction (PXRD)

Power X-ray diffraction (PXRD) experiments were performed using a PANalytical Empyrean powder X-ray diffractometer equipped with a monochromated Cu Kα X-ray source. Data was acquired between 3-80° 2θ at a scan rate of 1°/min. Data was analyzed using PANalytical HighScore software Version 4.8.

Powder X-ray diffraction (PXRD) analysis was conducted on the sample 1C (FIG. 1) and examples 2E-4E following calcination (FIGS. 2-4, respectively). By PXRD, all three samples were characterized as a doped molybdenum vanadium oxide phase known in academic literature as M1. This analysis shows that the method provided herein can produce a high crystalline purity of the catalytically active M1 phase.

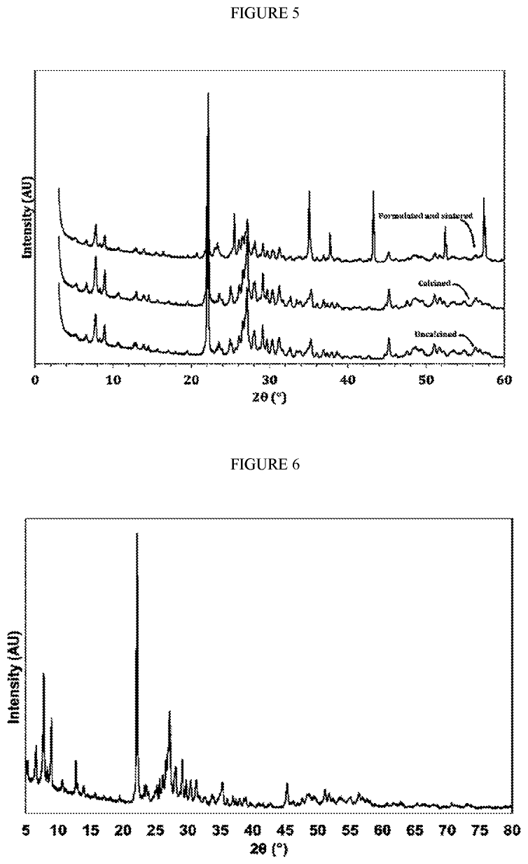

An overlay of the PXRD patterns of uncalcined, calcined and formulated/sintered catalyst 5E is shown in FIG. 5, and indicate that there is no apparent degradation of the M1 catalyst phase through the multiple heating cycles.

PXRD patterns of catalysts 6E and 7E are shown in FIGS. 6 and 7 respectively and also demonstrate the catalytically active M1 phase.

FIGS. 8 and 9 show an overlay of the PXRD patterns before and after calcination for examples 8E and 9E, respectively. In FIG. 9, minor peaks from unreacted MoO₃ were observed in the uncalcined sample. A listing of PXRD peak positions (° 2θ), calculated areas, and relative intensities for example 9E are presented in Table 10.

TABLE 10
PXRD Peak Listing for Example 9E.

| | Position | Area | Relative Intensity |
|---|---|---|---|
| Peak No. | [°2θ] | (calculated) | [%] |
| 1 | 5.2226 | 96.37 | 3.2 |
| 2 | 6.5578 | 184.45 | 7.51 |
| 3 | 7.7692 | 659.79 | 24.03 |
| 4 | 8.9161 | 291.58 | 14.67 |
| 5 | 10.6849 | 156.76 | 2.58 |
| 6 | 12.6294 | 14.67 | 1.97 |
| 7 | 12.956 | 77.54 | 2.93 |
| 8 | 13.9229 | 88.78 | 2.6 |
| 9 | 18.032 | 15.68 | 2.47 |
| 10 | 19.4426 | 15.69 | 1.07 |
| 11 | 22.1579 | 947.49 | 100 |
| 12 | 23.5609 | 79.43 | 4.52 |
| 13 | 23.8693 | 96.04 | 1.43 |
| 14 | 25.1114 | 117.17 | 3.07 |
| 15 | 26.1283 | 160.24 | 6.19 |
| 16 | 26.7037 | 348.46 | 8.95 |
| 17 | 27.1368 | 600.24 | 21.77 |
| 18 | 27.9053 | 98.63 | 12.97 |
| 19 | 28.1065 | 222.57 | 7.3 |
| 20 | 29.1211 | 278.4 | 14.98 |
| 21 | 29.7238 | 77.95 | 5.03 |
| 22 | 30.374 | 86.6 | 5.16 |
| 23 | 31.1889 | 118.68 | 1.6 |
| 24 | 31.272 | 220.85 | 5.66 |
| 25 | 32.5909 | 61.99 | 2.26 |
| 26 | 33.5151 | 14.43 | 1.29 |
| 27 | 33.9116 | 46.48 | 2.21 |
| 28 | 35.173 | 293.27 | 5.35 |
| 29 | 36.0756 | 19.46 | 1.97 |
| 30 | 36.9111 | 42.93 | 2.49 |
| 31 | 37.4008 | 23.9 | 1.64 |
| 32 | 37.9616 | 29.77 | 1.93 |
| 33 | 38.6733 | 72.31 | 2.2 |
| 34 | 39.9491 | 15.38 | 1.31 |
| 35 | 41.0446 | 38.14 | 0.96 |
| 36 | 41.5788 | 8.56 | 0.8 |
| 37 | 42.6863 | 25.67 | 1.25 |
| 38 | 45.2653 | 176.11 | 9.85 |
| 39 | 46.1227 | 25.18 | 1.21 |
| 40 | 47.5086 | 94.51 | 1.69 |
| 41 | 48.52 | 282.03 | 3.9 |
| 42 | 49.4759 | 208.49 | 2.67 |
| 43 | 51.0963 | 183.47 | 5.33 |
| 44 | 51.7602 | 88.17 | 3.46 |
| 45 | 52.3038 | 41.97 | 2.15 |
| 46 | 53.4348 | 221.69 | 2.81 |
| 47 | 54.8897 | 247.73 | 2.64 |
| 48 | 56.412 | 211.13 | 3.95 |
| 49 | 57.6189 | 202.06 | 2.84 |
| 50 | 60.5676 | 99.28 | 1.13 |
| 51 | 61.7123 | 41.21 | 0.94 |
| 52 | 62.8194 | 127.5 | 1.51 |
| 53 | 65.284 | 63.72 | 1.18 |
| 54 | 66.3128 | 74.04 | 1.03 |
| 55 | 67.9458 | 52.71 | 1.02 |
| 56 | 70.9927 | 89.43 | 0.97 |
| 57 | 73.2533 | 50.45 | 0.96 |

### Nitrogen Gas Sorption Analysis

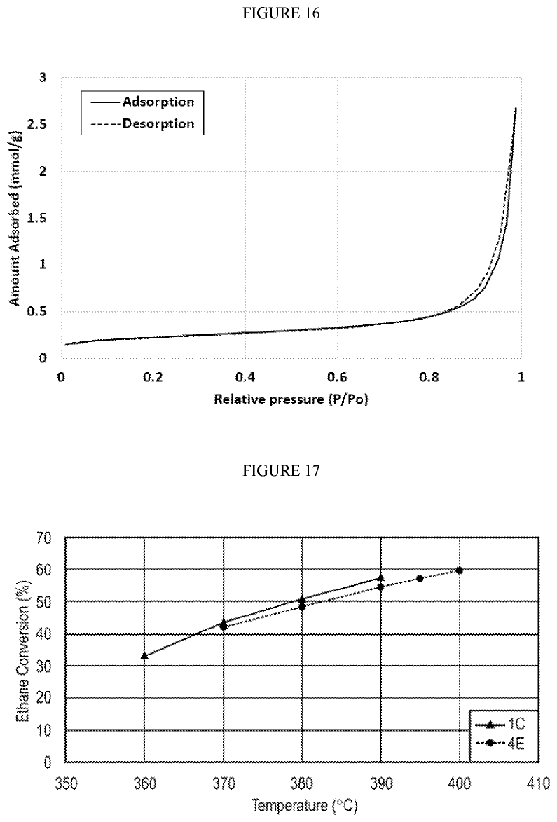

Nitrogen gas sorption analysis was conducted on a MICROMERITICS® TriStar instrument after degassing the samples under reduced atmosphere at 120° C. for 12 hours prior to analysis. Nitrogen sorption was conducted at 77 K using a liquid nitrogen bath. A nitrogen physisorption plot of example 9E is shown in FIG. 16.

Brunnauer-Emmett-Teller (BET) surface area analysis was applied to quantify the specific surface area (m²/g) of the solid samples. BET valuations were performed by multilayer adsorption of nitrogen and measured as a function of relative pressure. Applying BET analysis allows for quantitative comparison of the surface areas of solids by determining the monolayer capacity from nitrogen multilayer adsorption experiments. Monolayer capacity is a representation of total specific surface area and encompasses both the external area and the pore are of a porous solid. BET analysis was performed using the Micromeritics MicroActive software. Surface areas for examples 9E and 11E are found in Table 11.

TABLE 11
BET Surface Areas for Examples 9E and 11E.

| Example | BET Surface Area (m²/g) |
|---|---|
| 9E (uncalcined) | 55 |
| 9E (calcined) | 17 |
| 11E (uncalcined) | 56 |
| 11E (calcined) | 12 |

The Barrett-Joyner-Halenda (BJH) method was used for calculating the pore volume from experimentally collected adsorption isotherms using the Kelvin model of more filling (cm³/g). The BJH analysis was performed using the Micromeritics MicroActive software. A BJH plot of example 9E is shown in FIG. 15. Pore volumes for examples 9E and 11E are found in Table 12.

TABLE 12
Pore volumes for Examples 9E and 11E.

| Example | Pore Volume (cm³/g) |
|---|---|
| 9E (uncalcined) | 0.14 |
| 9E (calcined) | 0.10 |
| 11E (uncalcined) | 0.14 |
| 11E (calcined) | 0.06 |

### Thermogravimetric Analysis (TGA)

TGA was used for determining the water of hydration of the niobium and tantalum oxide starting materials. TGA was performed using a TA Instruments SDT650 thermogravimetric analyzer. Heating was performed at 2° C./min from 25-550° C. under N₂ flow.

### Crush Strength Testing

Crush strength of pelleted catalyst was tested using a Torbal FB Thor force gauge. Axial and radial crush strength measurements of cylindrical pellets were determined following ASTM D4179-22. Formulated catalyst materials (pelleted with, for example, a support or carrier) were observed to have axial crush strengths ranging from 90 to 120 N.

### Density (Envelope and Bulk)

Density was measured according to ASTM D3766. Envelope density was measured on 5-10 pellets at a time by individually measuring the pellet dimensions with a digital caliper, then weighing the pellet. Bulk density was measured by filling a graduated cylinder with pellets and measuring volume and mass, both before and after tapping to settle the pellets.

### Catalyst Testing

The catalysts described herein were tested for their ability to catalyze the oxidative dehydrogenation (ODH) of ethane using a microreactor unit (MRU). The MRU has a reactor tube made from stainless-steel SWAGELOK® Tubing, which had an outer diameter of 0.5 inches (1.27 cm), an internal diameter of 0.4 inches (1.02 cm), and a length of 13.4-15 inches (34.0-38.1 cm). Experimental temperatures of the MRU were measured using a 6-point WIKA Instruments Ltd. K-type thermocouple, which had an outer diameter of 0.125 inches (0.318 cm) and was inserted through the reactor. The 6-point thermocouple was used to measure and control the temperature within the catalyst bed. A room temperature stainless steel condenser was located after the reactor to collect water/acetic acid condensates. The gas product flow was either vented or was directed to an Agilent 8890 “hot gas” Gas Chromatograph (HGGC) during times when product gas analysis was required.

The samples were pressed into pellets using a steel die and hydraulic press, then the pellet was pulverized and particle sizes of 425-710 μm were sieved out for loading into the MRU. Approximately 2 g of sample was placed in the reactor. In the case of the formulated 50:50 catalyst:alumina pellets, approximately 4 g of sample was placed in the reactor. Once the catalyst bed was loaded into the reactor and connected to the MRU equipment, the testing was conducted as described herein.

The catalyst bed was loaded in the middle zone of the reactor and the remaining volume of the reactor was packed with quartz sand to produce the catalyst bed volume of 6 mL to ensure the catalyst volume was sufficient to cover the thermocouple area between points 2 and 5. The reactor loading was then secured with glass wool on both the top and the bottom of the reactor. Quartz sand was added to produce the catalyst bed volume of 6 mL to ensure the catalyst volume was sufficient to cover the thermocouple area.

The flow rate of the gas feed was adjusted to a target of 150 sccm (weight hour space velocity (WHSV)=3.57 h⁻¹). The target gas feed composition was 20 mol % ethane, 10 mol % oxygen and 70 mol % nitrogen for all testing. The target pressure was 20 psig. Gas composition was determined by gas chromatography (GC) using an Agilent 6890N Gas Chromatograph, and analyzed using Chrom Perfect—Analysis, Version 6.1.10 for data evaluation. Samples were left on stream at temperature between 38° and 420° C. until data appeared to equilibrate, which was approximately 5 days.

The mol. % ethane conversion temperature was determined at the WHSV of 3.57 h⁻¹, and a gas hourly space velocity (GHSV) in the range of 2000 to 5000 h⁻¹. The gaseous product exiting the catalyst bed was directed to vent during runs. When the gaseous product was to be analyzed, it was momentarily redirected to a gas chromatography unit to determine the percent of ethane, ethylene, O₂, CO₂, CO, and, optionally, acetic acid. The gas exiting the reactor was analyzed by gas chromatography. Conversion I of the ethane feed gas was calculated as a volume flow rate change of ethane in the product compared to feed ethane mass flow rate using the following formula:

$$C = ( \frac{2 * {X}_{Ethylene} + {X}_{C O 2} + {X}_{C O} + 2 * {X}_{Acetic Acid}}{2 * {X}_{Ethylene} + 2 * {X}_{Ethane} + {X}_{C O 2} + {X}_{C O} + 2 * {X}_{Acetic Acid}} ) * 100 \% \qquad Eq . 1$$

In Eq. 1, X is the molar concentration of the corresponding compound in the gaseous effluent exiting the reactor at corresponding temperature.

The gas exiting the reactor was analyzed by GC to determine catalyst or catalyst material selectivity to ethylene (i.e., the percentage on a molar basis of ethane that forms ethylene). Selectivity to ethylene (S_{Ethylene}) was determined using the following equation:

$${S}_{Ethylene} = ( \frac{2 * {X}_{Ethylene}}{2 * {X}_{Ethylene} + {X}_{CO 2} + {X}_{CO} + 2 * {X}_{Acetic Acid}} ) * 100 \% \qquad Eq . 2$$

In the above equation 2, S_{Ethylene} is the selectivity to ethylene and X is the molar concentration of the corresponding compound in the gaseous effluent exiting the reactor at corresponding temperature.

In order to close the mass balance for ODH experiments based on GC analysis of non-condensable products, it was assumed that all non-condensable gaseous products behave as ideal gases. The ideal gas equation of state is accurate in prediction of gas mixture behavior at operating pressure close to 1 atm absolute. For the ODH experiments, the product gas samples were collected and injected to a gas chromatograph (GC) at operating pressure close to 1 atm absolute. Therefore, the ideal gas behavior assumption is expected to generate accurate prediction of the gas mixture behavior. The bulk chemical reactions shown in Table 13 were assumed in order to calculate the amounts of condensable products. The reactions in Table 13 were used for the purpose of stoichiometrically-balanced mass balance calculations and not to represent the actual chemical reactions occurring in the ODH reaction.

TABLE 13
Bulk Chemical Reactions Assumed
for Mass Balance Methodology.

| | Assumption for the mass balance |
|---|---|
| Reaction | methodology |
| C₂H₆ + 0.5•O₂ → | One mole of water is produced per |
| C₂H₄ + H₂O | one mole of produced ethylene |
| C₂H₆ + 1.5•O₂ → | One mole of water is produced per |
| C₂H₄O₂ + H₂O | one mole of produced acetic acid |
| C₂H₆+ 3.5•O₂ → | Three moles of water are produced |
| 2•CO₂ + 3•H₂O | per two moles of produced CO₂ |
| C₂H₆ + 2.5•O₂ → | Three moles of water are produced |
| 2•CO + 3•H₂O | per two moles of produced CO |
| 2•CO₂ + 4•C₂H₆ → | Two moles of water are produced |
| C₂H₄O₂ + | for each two moles of CO₂ |
| 4•C₂H₄ + 2•H₂O | being consumed, to produce 1 mole |
| | of acetic acid and 4 moles of |
| | ethylene.^{a} |

^{a}As a result, the corresponding amount of water per mole of produced acetic acid and ethylene will be reduced. For example, 1 mole of acetic acid and 4 moles of ethylene would give 5 moles of water, when produced by reacting ethane and oxygen, but the same amount of both compounds would result in 2 moles of water for the same compounds to be produced by reaction of ethane and CO₂. This results in 3 moles less water produced to make these compounds for each 2 moles of CO₂ being consumed.
These will be subtracted in the mass balance.

Based on reactions shown in Table 13, the following method was programmed and used in MS Excel. A GRG Nonlinear solving method was used with the objective of setting the absolute deviation of estimated and measured oxygen from the reactor to zero by modifying the acetic acid output in the solver.

In step 1, the total molar flow of C₂ (ethane) into the reactor is calculated using Equation 3:

$${F}_{2 Total} = 100000 * {F}_{T otal} * ( {C}_{E t h a n e} + 0.5 * {C}_{C O 2} ) / 22.4 \qquad Eq . 3$$

wherein F_{2Total} is the total molar flow of C₂ into the reactor, [mmol/min]; F_{Total} is the total feed flow to reactor (including all diluents), [sccm]; C_{Ethane} is the molar fraction of ethane in total feed; C_{CO2} is the molar fraction of CO₂ in total feed; and 22.4 is the molar volume at STP, [l/mol].

In step 2, the molar flow of all reactive compounds in product effluent from the reactor is calculated excluding inert diluents.

The total molar flow of acetic acid in the product, [mmol/min](F_{AAout}) is estimated by Equation 4:

$${F}_{A A o u t} = y ( first estimate : y = 1 [ mmol / min ] \qquad Eq . 4$$

The total molar flows of C₂ in non-condensable compounds in the reactor product is calculated using Equation 5:

$${F}_{2 o u t x} = {F}_{2 Total} * ( {C}_{xout} / ( {SC}_{xout} ) ) * ( ( {F}_{2 Total} - {F}_{A A o u t} ) / {F}_{2 T otal} ) \qquad Eq . 5$$

wherein F₂ₒᵤₜₓ is the total molar flow of C₂ of x, [mmol/min]; Cₓₒᵤₜ is the molar fraction of x in the reactor product; and x is Ethane, Ethylene, CO₂, or CO.

The total molar flow of O₂ from the reactor is calculated using the following algorithm:

If F_{2OUTC-2}−(100000*F_{Total}*(0.5*C_{CO2})/22.4))>0 then use Equation 6:

$${F}_{O 2 o u t} = ( 100000 * {F}_{Total} * ( 0.5 * {C}_{O 2} ) / 22.4 ) ) - 0.5 * {F}_{2 o utetha - e} - 3.5 * {F}_{2 OUTCO 2} - 2.5 * {F}_{2 out - O} - 1.5 * {F}_{outAAout} \qquad Eq . 6$$

If F_{2OUTCO2}−(100000*F_{Total}*(0.5*C_{CO2})/22.4))<0 then use Equation 7:

$${F}_{O 2 out} = ( 100000 * {F}_{Total} * ( 0.5 * {C}_{O 2} ) / 22.4 ) ) - 0.5 * {F}_{2 outethane} + ABS ( 3.5 * {F}_{2 OUTCO -} ) - 2.5 * {F}_{2 out - O} - 1.5 * {F}_{outAAout} \qquad Eq . 7$$

The total molar flow of H₂O from the reactor is calculated using Equation 8:

$${F}_{H 2 O o u t x} = {F}_{H 2 O} + {F}_{2 o utetha - e} - 3 * {F}_{2 O UTCO 2} - 3 * {F}_{2 out - O} - {F}_{A A o u t} \qquad Eq . 8$$

In step 3, the molar fractions of all reactive compounds in the product effluent from the reactor are calculated on the dry (water free) basis, using F_{AAout} from step X04.

The molar fraction of acetic acid in the product is calculated using Equation 9:

$${C}_{A A o u tcalc} = {F}_{A A o u t} / ( {F}_{A A o u t} + S {F}_{xout} + {F}_{O 2 out} ) \qquad Eq . 9$$

The molar fraction of C₂ in ethane, ethylene, CO₂ and CO in the product is calculated using Equation 10:

$${C}_{xoutcalc} = {F}_{2 outx} / ( {F}_{A A o u t} + S {F}_{xout} + {F}_{O 2 o u t} ) \qquad Eq . 10$$

The molar fraction of oxygen in the product is calculated using Equation 11:

$${C}_{O 2 outcalc} = {F}_{O 2 o u t} / ( {F}_{A A o u t} + S {F}_{xout} + {F}_{O 2 out} ) \qquad Eq . 11$$

In step 4, the absolute deviation of estimated and measured O₂ in the non-condensable product from the reactor is calculated using Equation 12:

$${D}_{O 2} = {C}_{O 2 outcalc} - ( {C}_{O 2} / ( {C}_{Ethane} + {E}_{Ethylne} + 0.5 * {C}_{CO 2} + 0.5 {C}_{CO} + {C}_{O 2} ) * - 1 - {C}_{AAoutcalc} ) ) \qquad Eq . 12$$

Step 5 is the decision point, if D_{O2} is less than 10⁻⁴, proceed to step 6. If D_{O2} is not less than 10⁻⁴, return to step 2 and repeat. On the repeated steps, F_{AAout}=y (wherein y=new estimate [mmol/min]) is changed, and the steps are repeated to determine whether D_{O2} is closer to the target in step 410.

In step 6, ethane conversion is calculated using Equation 13:

$$X = ( \frac{2 * {C}_{Ethyleneoutcalc} + {C}_{CO 2 outcalc} + {C}_{COoutcalc} + 2 * {C}_{A A C a l c}}{\begin{matrix}2 * {C}_{EthyleneoutCalc} + 2 * {C}_{EthaneoutCalc} + {C}_{CO 2 outCalc} + \\ {C}_{COOutCalc} + 2 * {C}_{AACalc}\end{matrix}} ) * 100 \% \qquad Eq . 13$$

and selectivity toward each product is calculated using Equation 14:

$${S}_{x} = ( \frac{2 * {C}_{Xoutcalc}}{\begin{matrix}2 * {C}_{EthyleneoutCalc} + 2 * {C}_{EthaneoutCalc} + {C}_{CO 2 outCalc} + \\ {C}_{COoutCalc} + 2 * {C}_{AAoutCalc}\end{matrix}} ) * 100 \% \qquad Eq . 14$$

wherein C_{xoutCalc}=C_{EthyleneoutCalc}, C_{AAoutCalc}, 0.5*C_{CO2outCalc} or 0.5*C_{COoutcalc}.

### Summary of Catalyst Performance

The results for comparative sample 1C and samples 4E, 5E, 6E, 7E, 8E, 9E, and 10E are summarized in Table 14.

TABLE 14
Catalyst Performance.

| | | | Ethane | Ethylene | Temper- |
|---|---|---|---|---|---|
| | Catalyst | Flow | Conversion | Selectivity | ature |
| | Mass (g) | (sccm) | (%) | (%) | (° C.) |
| 1C | 2.0146 | 153 | 50.5 | 87.0 | 380 |
| 4E | 2.0003 | 152 | 48.3 | 91.2 | 380 |
| 5E | 2.0128 | 153 | 52.3 | 85.0 | 400 |
| 5E-Pelleted | 1.9980 | 152 | 52.7 | 83.5 | 405 |
| 6E | 2.0053 | 153 | 26.4 | 89.9 | 360 |
| 6E | 2.0053 | 153 | 43.5 | 83.5 | 392 |
| 7E | 1.9983 | 152 | 21.1 | 88.4 | 390 |
| 8E | 2.0040 | 152 | 40.1 | 92.2 | 350 |
| 8E | 2.0040 | 152 | 46.0 | 91.0 | 360 |
| 8E | 2.0040 | 152 | 49.2 | 90.2 | 365 |
| 9E | 1.9995 | 153 | 47.7 | 92.4 | 370 |
| 9E | 1.9995 | 153 | 60.1 | 90.0 | 390 |
| 10E | 2.0090 | 153 | 48.6 | 85.7 | 357 |

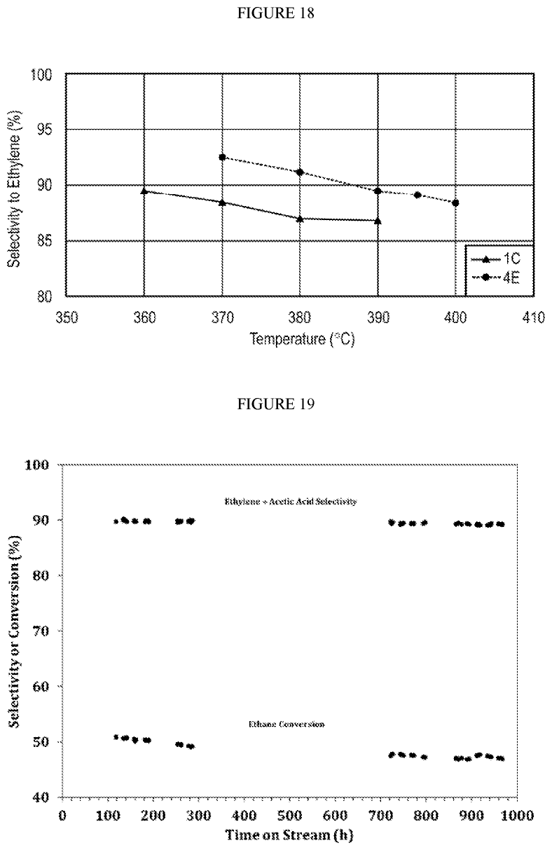

FIG. 17 is a plot of the conversion of ethane for comparative catalyst 1C and sample 4E. FIG. 18 is a plot of the selectivity of ethylene formation for comparative catalyst 1C and sample 4E.

As shown in Table 14 and FIG. 17, sample 4E that includes a bismuth dopant has comparable activity to sample 1C that includes a tellurium dopant. As shown in Table 14 and FIG. 18, sample 4E has improved selectivity to ethylene compared to sample 1C. The data in Table 14 also shows that increasing the amount of bismuth hydroxide in the synthesis may have a detrimental impact on catalyst selectivity. The data also suggests that not washing the catalyst sample after synthesis may lower the activity of the catalyst but appears to only have a minor impact on selectivity. Additionally, lowering the amount of tantalum may have a beneficial effect on both activity and selectivity. Further, a simplified synthesis omitting the solid grinding step provided a catalyst with good activity and selectivity.

Longer term testing was performed on the formulated catalyst 5E-pelleted at 405° C. for a total of 1032 hours. As shown in FIG. 19 and Table 15 there was acceptable loss in performance over this time. Data points were excluded if pressure deviated significantly from 23.5±1.0 psig, as catalyst activity has a strong positive correlation to pressure.

TABLE 15
Comparison of Initial and Final Catalyst Performance of Formulated
Mo VₐTa_{b}Bi_{c}Oₓ (5E-Pelleted) in Long Term Testing.

| | Ethane | Ethylene | Acetic Acid | COₓ | Temper- |
|---|---|---|---|---|---|
| | Conver- | Selectivity | Selectivity | Selectivity | ature |
| | sion (%) | (%) | (%) | (%) | (° C.) |
| 118 h on | 52.7 | 83.5 | 4.0 | 9.5 | 405 |
| stream | | | | | |
| 1032 h on | 48.0 | 83.8 | 6.2 | 10 | 405 |
| stream | | | | | |

Non-limiting embodiments of the present disclosure include the following:

Embodiment A. A catalyst comprising the formula MoₐV_{b}Bi_{c}M_{d}Oₓ, wherein M is Ta or Nb, or a mixture thereof, a is 1.0; b is 0.01 to 0.5; c is 0.005 to 0.2; d is 0.005 to 0.1; and x is the number of oxygen atoms necessary to render the catalyst electrically neutral; and wherein a, b, c, and d are determined based on the amount of each starting material used to form the catalyst.

Embodiment B. The catalyst of Embodiment A, wherein the values of a, b, c, and d are also determined by elemental analysis.

Embodiment C. The catalyst of Embodiment A or B, wherein b is 0.2 to 0.4; c is 0.01 to 0.07; and d is 0.01 to 0.07.

Embodiment D. The catalyst of claim Embodiment A, B, or C, wherein b is 0.30 to 0.35; c is 0.04 to 0.05; and d is 0.03 to 0.05.

Embodiment E. The catalyst of Embodiment, A, B, or C, wherein b is 0.2 to 0.3; c is 0.05 to 0.07; and d is 0.02 to 0.04.

Embodiment F. The catalyst of Embodiment A, B, or C, wherein b is 0.3 to 0.4; c is 0.05 to 0.07; and d is 0.03 to 0.05.

Embodiment G. The catalyst of Embodiment A, B, or C, wherein the catalyst has a formula selected from Mo₁V_{0.31}Bi_{0.04}M_{0.05}Oₓ, Mo₁V_{0.31}Bi_{0.05}M_{0.05}Oₓ, Mo₁V_{0.31}Bi_{0.06}M_{0.05}Oₓ, Mo₁V_{0.32}Bi_{0.05}M_{0.05}Oₓ, Mo₁V_{0.32}Bi_{0.04}M_{0.03}Oₓ, Mo₁V_{0.33}Bi_{0.05}M_{0.04}Oₓ, Mo₁V_{0.26}Bi_{0.06}M_{0.03}Oₓ, Mo₁V_{0.33}Bi_{0.06}M_{0.04}Oₓ, and Mo₁V_{0.26}Bi_{0.05}M_{0.05}Oₓ.

Embodiment H. The catalyst of Embodiment A, B, or C, wherein the catalyst has a formula selected from Mo₁V_{0.32}Bi_{0.04}Ta_{0.03}Oₓ, Mo₁V_{0.33}Bi_{0.05}Ta_{0.04}Oₓ, Mo₁V_{0.26}Bi_{0.06}Ta_{0.03}Oₓ, Mo₁V_{0.31}Bi_{0.06}Nb_{0.01}Oₓ, Mo₁V_{0.33}Bi_{0.06}Ta_{0.04}Oₓ, and Mo₁V_{0.26}Bi_{0.05}Ta_{0.05}Oₓ.

Embodiment I. The catalyst of Embodiment A, B, C, or H, wherein the catalyst has a formula selected from: Mo₁V_{0.32}Bi_{0.04}Ta_{0.03}Oₓ, Mo₁V_{0.33}Bi_{0.05}Ta_{0.04}Oₓ, Mo₁V_{0.26}Bi_{0.05}Ta_{0.05}Oₓ, and Mo₁V_{0.26}Bi_{0.06}Ta_{0.03}Oₓ, wherein the formula is determined by energy dispersive X-ray spectroscopy (EDX).

Embodiment J. The catalyst of Embodiment A, B, C, D, E, F, G, H, or I, having a pore volume from 0.02 cm³/g to 0.25 cm³/g, as determined by a nitrogen physisorption analysis with a Barrett-Joyner-Halenda (BJH) model.

Embodiment K. The catalyst of Embodiment A, B, C, D, E, F, G, H, I, or J, having a pore volume from 0.1 cm³/g to 0.2 cm³/g, as determined by a nitrogen physisorption analysis with a Barrett-Joyner-Halenda (BJH) model.

Embodiment L. The catalyst of Embodiment A, B, C, D, E, F, G, H, I, J, or K, having a Brunauer-Emmett-Teller (BET) surface area from 5 m²/g to 60 m²/g, as determined by a nitrogen physisorption analysis.

Embodiment M. The catalyst of Embodiment A, B, C, D, E, F, G, H, I, J, K, or L, having a Brunauer-Emmett-Teller (BET) surface area from 10 m²/g to 25 m²/g, as determined by a nitrogen physisorption analysis.

Embodiment N. A catalyst material comprising the catalyst of Embodiment A, B, C, D, E, F, G, H, I, J, K, L, or M, and a catalyst support or carrier.

Embodiment O. The catalyst material Embodiment N, wherein the catalyst support or carrier is selected from the group consisting of precipitated synthetic silica, fumed synthetic silica, silica-alumina, α-alumina, γ-alumina, titania, WO₃—ZrO₂, silicon carbide, MgAl spinel, calcium aluminate, zirconia and boron nitride.

Embodiment P. The catalyst of Embodiment N or O, wherein the catalyst support or carrier is α-alumina.

Embodiment Q. The catalyst of Embodiment A, B, C, D, E, F, G, H, I, J, K, L, or M, wherein the catalyst has an ethylene selectivity of at least 90% at an ethane conversion of at least 45% in an oxidative dehydrogenation reaction of ethane.

Embodiment R. The catalyst of Embodiment A, B, C, D, E, F, G, H, I, J, K, L, or M, wherein the catalyst has a 45% ethane conversion temperature from 300° C. to 420° C. in an oxidative dehydrogenation reaction of ethane.

Embodiment 5. The catalyst of Embodiment A, B, C, D, E, F, G, H, I, J, K, L, or M, wherein the catalyst has a 45% ethane conversion temperature from 350° C. to 400° C. in an oxidative dehydrogenation reaction of ethane.

Embodiment T. The catalyst material of Embodiment N or O, having an ethylene selectivity of at least 90% at an ethane conversion of at least 45% in an oxidative dehydrogenation reaction of ethane.

Embodiment U. The catalyst material of Embodiment N or O, having a 45% ethane conversion temperature from 300° C. to 420° C. in an oxidative dehydrogenation reaction of ethane.

Embodiment V. The catalyst material of Embodiment N or O, having a 45% ethane conversion temperature from 300° C. to 420° C. in an oxidative dehydrogenation reaction of ethane.

Embodiment W. A method for preparing a catalyst comprising forming a slurry comprising metal oxides, a bismuth compound, a reducing agent, and water; and heating the slurry to form the catalyst; wherein the metal oxides comprise an oxide of molybdenum; an oxide of vanadium; and an oxide of tantalum or an oxide of niobium, or both; and wherein the bismuth compound comprises bismuth oxide, bismuth hydroxide, or a bismuth carbonate.

Embodiment X. The method according to Embodiment W, wherein a ratio of the water in the slurry to amount of catalyst formed is between 0.1 mL water per gram of catalyst and 10 mL water per gram of catalyst.

Embodiment Y. The method according to Embodiment W or X, wherein the ratio of the water in the slurry to amount of catalyst formed between 0.2 mL water per gram of catalyst and 1 mL water per gram of catalyst.

Embodiment Z. The method according to Embodiment W, X, or Y, wherein the slurry has a ratio of water to metal oxides between 0.2 mL water per gram of metal oxides and 0.6 mL water per gram of metal oxides.

Embodiment AA. The method according to Embodiment W, X, Y, or Z, wherein the slurry has a ratio of water to metal oxides between 0.3 mL water per gram of metal oxides and 0.5 mL water per gram of metal oxides.

Embodiment AB. The method according to Embodiment W, X, Y, Z, or AA, wherein the oxide of molybdenum is MoO₃.

Embodiment AC. The method according to Embodiment W, X, Y, Z, AA, or AB, wherein the oxide of vanadium is V₂O₅.

Embodiment AD. The method according to Embodiment W, X, Y, Z, AA, AB, or AC, wherein the tantalum oxide, when present, is Ta₂O₅·xH₂O, and the niobium oxide, when present, is Nb₂O₅·H₂O.

Embodiment AE. The method according to Embodiment W, X, Y, Z, AA, AB, AC, or AD, wherein the bismuth compound is bismuth hydroxide.

Embodiment AF. The method according to Embodiment W, X, Y, Z, AA, AB, AC, AD, or AE, further comprising grinding, wet milling, dry milling, or crushing the metal oxides and the bismuth compound.

Embodiment AG. The method according to Embodiment W, X, Y, Z, AA, AB, AC, AD, AE, or AF, further comprising grinding, wet milling, dry milling, or crushing the reducing agent.

Embodiment AH. The method according to Embodiment W, X, Y, Z, AA, AB, AC, AD, AE, AF, or AG, wherein the reducing agent comprises an alcohol, a carboxylic acid, or an ester.

Embodiment AI. The method according to Embodiment W, X, Y, Z, AA, AB, AC, AD, AE, AF, AG, or AH, wherein the reducing agent is oxalic acid.

Embodiment AJ. The method according to Embodiment W, X, Y, Z, AA, AB, AC, AD, AE, AF, AG, AH, or AI, wherein the slurry comprises no more than one reducing agent.

Embodiment AK. The method according to Embodiment W, X, Y, Z, AA, AB, AC, AD, AE, AF, AG, AH, AI, or AJ, comprising heating the slurry by ramping a temperature from ambient to a temperature between 100° C. and 200° C. over a ramping time between 2 hours and 48 hours; and holding the temperature at a holding temperature between 100° C. and 200° C. for a holding time between 12 hours and 160 hours.

Embodiment AL. The method according to Embodiment W, X, Y, Z, AA, AB, AC, AD, AE, AF, AG, AH, AI, or AJ, comprising heating the slurry by ramping a temperature from ambient to a temperature between 100° C. and 200° C. over a ramping time between 2 hours and 48 hours; and holding the temperature at a holding temperature between 100° C. and 200° C. for a holding time between 12 hours and 120 hours.

Embodiment AM. The method according to W, X, Y, Z, AA, AB, AC, AD, AE, AF, AG, AH, AI, AJ, AK, or AL, further comprising washing the catalyst with water.

Embodiment AN. The method according to W, X, Y, Z, AA, AB, AC, AD, AE, AF, AG, AH, AI, AJ, AK, AL, or AM further comprising calcining the catalyst to form a calcined catalyst.

Embodiment AO. The method according to Embodiment AN, comprising calcining the catalyst by placing the catalyst in a furnace under an oxygen-free environment; ramping a temperature of the furnace from ambient to a temperature between 500° C. and 620° C. over a ramping time between 2 hours and 10 hours; and holding the temperature of the furnace at a holding temperature between 500° C. and 620° C. for a holding time between 1 hour and 10 hours.

Embodiment AP. The method according to W, X, Y, Z, AA, AB, AC, AD, AE, AF, AG, AH, AI, AJ, AK, AL, AM, AN, or AO, wherein the catalyst comprises the formula MoₐV_{b}Bi_{c}M_{d}Oₓ, wherein M is Ta or Nb, or a mixture thereof, a is 1.0; b is 0.01 to 0.5; c is 0.005 to 0.2; d is 0.005 to 0.1; and x is the number of oxygen atoms necessary to render the catalyst electrically neutral; wherein a, b, c, and d are determined based on the amount of each starting material used to form the catalyst.

Embodiment AQ. The method according to Embodiment AP, wherein the values of a, b, c, and d are also determined by elemental analysis.

Embodiment AR. The method according to claim Embodiment AP or AQ wherein the catalyst has a formula selected from Mo₁V_{0.31}Bi_{0.04}M_{0.05}Oₓ, Mo₁V_{0.31}Bi_{0.05}M_{0.05}Oₓ, Mo₁V_{0.31}Bi_{0.06}M_{0.05}Oₓ, Mo₁V_{0.32}Bi_{0.05}M_{0.05}Oₓ, Mo₁V_{0.32}Bi_{0.04}M_{0.03}Oₓ, Mo₁V_{0.33}Bi_{0.05}M_{0.04}Oₓ, Mo₁V_{0.26}Bi_{0.06}M_{0.03}Oₓ, Mo₁V_{0.33}Bi_{0.06}M_{0.04}Oₓ, and Mo₁V_{0.26}Bi_{0.05}M_{0.05}Oₓ.

Embodiment AS. The method according to claim AP or AQ, wherein the catalyst has a formula selected from Mo₁V_{0.32}Bi_{0.04}Ta_{0.03}Oₓ, Mo₁V_{0.33}Bi_{0.05}Ta_{0.04}Oₓ, Mo₁V_{0.26}Bi_{0.06}Ta_{0.03}Oₓ, Mo₁V_{0.31}Bi_{0.06}Nb_{0.03}Oₓ, Mo₁V_{0.33}Bi_{0.06}Ta_{0.04}Oₓ, and Mo₁V_{0.26}Bi_{0.05}Ta_{0.05}Oₓ.

Embodiment AT. The method according to Embodiment W, X, Y, Z, AA, AB, AC, AD, AE, AF, AG, AH, AI, AJ, AK, AL, AM, AN, AO, AP, AQ, AR, or AS, wherein the metal oxides and the bismuth compound each have a particle size in the range of from 0.5 μm to 250 μm.

Embodiment AU. The method according to Embodiment X, Y, Z, AA, AB, AC, AD, AE, AF, AG, AH, AI, AJ, AK, AL, AM, AN, AO, AP, AQ, AR, AS, or AT, wherein a ratio of a total amount of water used to prepare the catalyst is between 0.2 mL water per gram of catalyst and 25 mL water per gram of catalyst.

Embodiment AV. The method of Embodiment X, Y, Z, AA, AB, AC, AD, AE, AF, AG, AH, AI, AJ, AK, AL, AM, AN, AO, AP, AQ, AR, AS, AT, or AU, wherein the catalyst has an ethylene selectivity of at least 90% at an ethane conversion of at least 45% in an oxidative dehydrogenation reaction of ethane.

Embodiment AW. A process for oxidative dehydrogenation of ethane, the process comprising contacting a gaseous feed comprising ethane and oxygen with a catalyst in a reactor to produce an effluent comprising ethylene, wherein the catalyst has the formula MoₐV_{b}Bi_{c}M_{d}Oₓ, wherein M is Ta or Nb, or a mixture thereof, a is 1.0; b is 0.01 to 0.5; c is 0.005 to 0.2; d is 0.005 to 0.1; and x is the number of oxygen atoms necessary to render the catalyst electrically neutral; and wherein a, b, c, and d are determined based on the amount of each starting material used to form the catalyst.

Embodiment AX. The process of Embodiment AW, wherein the values of a, b, c, and d are also determined by elemental analysis.

Embodiment AY. The process of Embodiment AW or AX, wherein b is 0.2 to 0.4; c is 0.01 to 0.07; and d is 0.01 to 0.07.

Embodiment AZ. The process of Embodiment AW, AX, or AY, wherein b is 0.30 to 0.35; c is 0.04 to 0.05; and d is 0.03 to 0.05.

Embodiment AAA. The process of Embodiment AW, or AX, wherein b is 0.2 to 0.3; c is 0.05 to 0.07; and d is 0.02 to 0.04.

Embodiment AAB. The process of Embodiment AW or AX, wherein b is 0.3 to 0.4; c is 0.05 to 0.07; and d is 0.03 to 0.05.

Embodiment AAC. The process of Embodiment AW, AX, or AY, wherein the catalyst has a formula selected from Mo₁V_{0.31}Bi_{0.04}M_{0.05}Oₓ, Mo₁V_{0.31}Bi_{0.05}M_{0.05}Oₓ, Mo₁V_{0.31}Bi_{0.06}M_{0.05}Oₓ, Mo₁V_{0.32}Bi_{0.05}M_{0.05}Oₓ, Mo₁V_{0.32}Bi_{0.04}M_{0.03}Oₓ, Mo₁V_{0.33}Bi_{0.05}M_{0.04}Oₓ, Mo₁V_{0.26}Bi_{0.06}M_{0.03}Oₓ, Mo₁V_{0.33}Bi_{0.06}M_{0.04}Oₓ, and Mo₁V_{0.26}Bi_{0.06}M_{0.03}Oₓ.

Embodiment AAD. The process of Embodiment AW, AX, or AAY, wherein the catalyst has a formula selected from Mo₁V_{0.32}Bi_{0.04}Ta_{0.03}Oₓ, Mo₁V_{0.33}Bi_{0.05}Ta_{0.04}Oₓ, Mo₁V_{0.26}Bi_{0.06}Ta_{0.03}Oₓ, Mo₁V_{0.31}Bi_{0.06}Nb_{0.01}Oₓ, Mo₁V_{0.33}Bi_{0.06}Ta_{0.04}Oₓ, and Mo₁V_{0.26}Bi_{0.05}Ta_{0.05}Oₓ.

Embodiment AAE. The process of Embodiment AW, AX, AY, AZ, AAA, AAB, AAC, or AAD, wherein the catalyst is comprised in a catalyst material, the catalyst material including a catalyst support or carrier.

Embodiment AAF. The process of Embodiment AAE, wherein the catalyst support or carrier is α-alumina.

Embodiment AAG. The process of Embodiment AW, AX, AY, AZ, AAA, AAB, AAC, AAD, AAE, or AAF, having a 45% ethane conversion temperature from 300° C. to 420° C.

Embodiment AAH. The process of Embodiment AW, AX, AY, AZ, AAA, AAB, AAC, or AAD, AAE, or AAF, having a 45% ethane conversion temperature from 350° C. to 400° C.

Embodiment AAI. The process of Embodiment AW, AX, AY, AZ, AAA, AAB, AAC, or AAD, AAE, AAF, AAG, or AAH, further comprising converting the ethylene to a product.

Embodiment AAJ. The process of Embodiment AAI, wherein the product is a polyethylene is selected from very low density polyethylene (VLDPE), low density polyethylene (LDPE), linear low density polyethylene (LLDPE), medium density polyethylene (MDPE), and high density polyethylene (HDPE).

Other implementations are also within the scope of the following claims.

1. A catalyst comprising the formula: 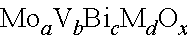
wherein:
M is Ta or Nb, or a mixture thereof,
a is 1.0;
b is 0.01 to 0.5;
c is 0.005 to 0.2;
d is 0.005 to 0.1; and
x is the number of oxygen atoms necessary to render the catalyst electrically neutral;
wherein a, b, c, and d are determined based on the amount of each starting material used to form the catalyst.

2. The catalyst of claim 1, wherein the values of a, b, c, and d are also determined by elemental analysis.

3. The catalyst of claim 1, wherein:
b is 0.2 to 0.4;
c is 0.01 to 0.07; and
d is 0.01 to 0.07.

4.-6. (canceled)

7. The catalyst of claim 1, wherein the catalyst has a formula selected from the group consisting of: Mo₁V_{0.31}Bi_{0.04}M_{0.05}Oₓ, Mo₁V_{0.31}Bi_{0.05}M_{0.05}Oₓ, Mo₁V_{0.31}Bi_{0.06}M_{0.05}Oₓ, Mo₁V_{0.32}Bi_{0.05}M_{0.05}Oₓ, Mo₁V_{0.32}Bi_{0.04}M_{0.03}Oₓ, Mo₁V_{0.33}Bi_{0.05}M_{0.04}Oₓ, Mo₁V_{0.26}Bi_{0.06}M_{0.03}Oₓ, Mo₁V_{0.33}Bi_{0.06}M_{0.04}Oₓ, and Mo₁V_{0.26}Bi_{0.05}M_{0.05}Oₓ.

8.-9. (canceled)

10. The catalyst of claim 1, having a pore volume between 0.02 cm³/g and 0.25 cm³/g, as determined by a nitrogen physisorption analysis with a Barrett-Joyner-Halenda (BJH) model.

11. The catalyst of claim 1, having a pore volume between 0.1 cm³/g and 0.2 cm³/g, as determined by a nitrogen physisorption analysis with a Barrett-Joyner-Halenda (BJH) model.

12. The catalyst of claim 1, having a Brunauer-Emmett-Teller (BET) surface area between 5 m²/g and 60 m²/g, as determined by a nitrogen physisorption analysis.

13. (canceled)

14. A catalyst material comprising the catalyst of claim 1 and a catalyst support or carrier.

15. The catalyst material of claim 14, wherein the catalyst support or carrier is selected from the group consisting of precipitated synthetic silica, fumed synthetic silica, silica-alumina, α-alumina, γ-alumina, titania, WO₃—ZrO₂, silicon carbide, MgAl spinel, calcium aluminate, zirconia, and boron nitride.

16. (canceled)

17. The catalyst of claim 1, wherein the catalyst has at least one of:
an ethylene selectivity of at least 90% at an ethane conversion of at least 45% in an oxidative dehydrogenation reaction of ethane;
a 45% ethane conversion temperature from 300° C. to 420° C. in the oxidative dehydrogenation reaction of ethane: or
a 45% ethane conversion temperature from 350° C. to 400° C. in an oxidative dehydrogenation reaction of ethane.

18.-22. (canceled)

23. A method for preparing a catalyst comprising:
forming a slurry comprising metal oxides, a bismuth compound, a reducing agent, and water; and
heating the slurry to form the catalyst;
wherein the metal oxides comprise:
an oxide of molybdenum;
an oxide of vanadium; and
an oxide of tantalum or an oxide of niobium, or both; and
wherein the bismuth compound comprises bismuth oxide, bismuth hydroxide, or a bismuth carbonate.

24. The method according to claim 23, wherein a ratio of the water in the slurry to amount of catalyst formed is between 0.1 mL water per gram of catalyst and 10 mL water per gram of catalyst.

25. (canceled)

26. The method according to claim 23, wherein the slurry has a ratio of water to metal oxides between 0.2 mL water per gram of metal oxides and 0.6 mL water per gram of metal oxides.

27. (canceled)

28. The method according to claim 23, wherein:
the oxide of molybdenum is MoO₃;
the oxide of vanadium is V₂O₅;
the tantalum oxide, when present, is Ta₂O₅·xH₂O;
the niobium oxide, when present, is Nb₂O₅·xH₂O; and
the bismuth compound is bismuth hydroxide.

29.-31. (canceled)

32. The method according to claim 23, further comprising grinding, wet milling, dry milling, or crushing the metal oxides, the reducing agent, and the bismuth compound.

33. (canceled)

34. The method according to claim 23, wherein the reducing agent comprises an alcohol, a carboxylic acid, or an ester.

35.-36. (canceled)

37. The method according to claim 23, comprising heating the slurry by:
ramping a temperature from ambient to a temperature between 100° C. and 200° C. over a ramping time between 2 hours and 48 hours; and
holding the temperature at a holding temperature between 100° C. and 200° C. for a holding time between 12 hours and 160 hours.

38.-48. (canceled)

49. A process for oxidative dehydrogenation of ethane, the process comprising contacting a gaseous feed comprising ethane and oxygen with a catalyst in a reactor to produce an effluent comprising ethylene, wherein the catalyst has the formula: 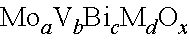
wherein:
M is Ta or Nb, or a mixture thereof,
a is 1.0;
b is 0.01 to 0.5;
c is 0.005 to 0.2;
d is 0.005 to 0.1; and
x is the number of oxygen atoms necessary to render the catalyst electrically neutral;
wherein a, b, c, and d are determined based on the amount of each starting material used to form the catalyst.

50.-60. (canceled)

61. The process of claim 49, further comprising converting the ethylene to a product.

62. The process of claim 61, wherein the product is a polyethylene is selected from very low density polyethylene (VLDPE), low density polyethylene (LDPE), linear low density polyethylene (LLDPE), medium density polyethylene (MDPE), and high density polyethylene (HDPE).
